(19) United States
(12) Patent Application Publication
(10) Pub. No.: US 2026/0020469 A1
(43) Pub. Date: Jan. 15, 2026

(54) DISPLAY APPARATUS

(71) Applicant: LG Display Co., Ltd., Seoul (KR)

(72) Inventor: Intae KO, Paju-si (KR)

(73) Assignee: LG Display Co., Ltd., Seoul (KR)

(21) Appl. No.: 19/217,514

(22) Filed: May 23, 2025

(30) Foreign Application Priority Data
Jul. 15, 2024 (KR) 10-2024-0092839

(51) Int. Cl. H10K 59/60; H10K 59/121; H10K 59/122; H10K 59/123; H10K 59/131; H10K 59/80

(52) U.S. Cl. CPC H10K 59/60; H10K 59/1213; H10K 59/122; H10K 59/123; H10K 59/131; H10K 59/8731; H10K 59/879

(57) ABSTRACT

A display apparatus according to one embodiment of the present specification includes a substrate including a display area including a plurality of pixels and a transmissive area between adjacent plurality of pixels, and a non-display area surrounding the display area. A thin film transistor is on the substrate, with a first protective layer on the thin film transistor. A connection electrode electrically connected to the thin film transistor is on the first protective layer, a second protective layer on the connection electrode. A light-emitting part is on the second protective layer, and a first microlens is on the light-emitting part. The first microlens is disposed in the transmissive area.

### CROSS REFERENCE TO RELATED APPLICATION

The present application claims priority to Korean Patent Application No. 10-2024-0092839, filed Jul. 15, 2024, the entire contents of which is incorporated herein for all purposes by this reference.

### BACKGROUND

### Technical Field

The present specification relates to a display apparatus.

### Description of the Related Art

As the information society develops, various demands for display apparatuses for displaying images are increasing, and various types of display apparatuses, such as a liquid crystal display (LCD) apparatus and an organic light emitting diode (OLED) display apparatus, are being utilized.

Among the display apparatuses, there is an advantage in that the OLED display apparatus as the self-luminous type has a wider viewing angle and a high contrast ratio, and is lighter and thinner and has less power consumption than the LCD apparatus because it does not require a separate backlight. In addition, there is an advantage in that the OLED display apparatus can drive at a low voltage, have a fast response time, and especially have the inexpensive manufacturing cost.

The OLED display apparatus can also be applied to display apparatuses mounted on vehicles. Among display apparatuses installed on a vehicle, display apparatuses in front of a driver's seat and a front passenger's seat need to limit a viewing angle of a driver according to driving situations of the driver. The display apparatus needs to limit a viewing angle according to a user's needs for privacy and information protection.

### BRIEF SUMMARY

The display apparatus integrates a light-receiving sensor beneath the display panel without requiring a through-hole. This is accomplished by forming transmissive areas between adjacent pixels and placing a first microlens in the transmissive area to focus external light onto the sensor. The design maintains the integrity and layout of the panel, improving manufacturability, durability, and overall aesthetics while enabling efficient light transmission.

A second microlens is positioned over each subpixel to control the emission direction of light, enhancing the viewing angle and supporting privacy-focused display functions. The transmissive area includes high-transmittance materials, avoiding opaque elements like transistors and electrodes, which increases light throughput and allows for flexible placement of sensors in both display and non-display areas.

Various embodiments of the present specification is directed to providing a display apparatus having a design with improved aesthetic feeling.

Various embodiments of the present specification is directed to providing a display apparatus in which a location of a light-receiving sensor can be freely disposed in a display panel.

Various embodiments of the present specification is directed to providing a display apparatus in which a separate through hole for light transmission can be unnecessary in a display panel.

Various embodiments of the present specification is directed to providing a display apparatus in which, since a through hole for light transmission is not formed in the display panel, a change in arrangement and shapes of circuit components, etc., of a display panel can be unnecessary.

Various embodiments of the present specification is directed to providing a display apparatus in which, even when a through hole for light transmission is not formed in a display panel, the amount of light passing through the display panel can be increased.

Technical benefits of the present specification are not limited to the above-described benefits, and other technical benefits may be inferred from the following embodiments.

According to one embodiment of the present specification, there is provided a display apparatus including a substrate including a display area including a plurality of pixels and a transmissive area between adjacent plurality of pixels, and a non-display area surrounding the display area, a thin film transistor disposed on the substrate, a first protective layer on the thin film transistor, a connection electrode electrically connected to the thin film transistor on the first protective layer, a second protective layer on the connection electrode, a light-emitting part on the second protective layer, and a first microlens on the light-emitting part, wherein the first microlens is disposed in the transmissive area.

According to another embodiment of the present specification, there is provided a display apparatus including a substrate including a display area including a plurality of pixels and a transmissive area between adjacent pixels and a non-display area around the display area, a thin film transistor disposed on the substrate, a first protective layer on the thin film transistor, a light-emitting part on the first protective layer, a first microlens on the light-emitting part, and a light-receiving sensor disposed under the substrate and provided as a plurality of light-receiving sensors, wherein the first microlens is disposed in the transmissive area, and at least a part of the light-receiving sensor overlaps the transmissive area.

Detailed matters of other embodiments are included in the detailed description and accompanying drawings.

According to the embodiments of the present specification, it is possible to provide the display apparatus with improved aesthetic feeling.

According to the embodiments of the present specification, the location of the light-receiving sensor can be freely disposed in the display panel.

According to the embodiments of the present specification, a separate through hole for light transmission can be unnecessary in the display panel.

According to the embodiments of the present specification, since the through hole for light transmission is not formed in the display panel, a change in arrangement and shapes of circuit components, etc., of the display panel can be unnecessary.

According to the embodiments of the present specification, even when the through hole for light transmission is not formed in the display panel, the amount of light passing through the display panel can be increased.

According to the embodiments of the present specification, since the through hole for light transmission is unnecessary in the display panel, it is possible to maintain the arrangement and shapes of circuit components, etc., of the display panel, thereby more efficiently performing the process of the display apparatus and reducing production energy.

However, effects obtainable from the present specification are not limited to the above-described effects, and other effects that are not mentioned will be able to be clearly understood by those skilled in the art to which the present specification pertains based on the following description.

### BRIEF DESCRIPTION OF THE SEVERAL VIEWS OF THE DRAWINGS

FIG. 1 is a plan view of a display apparatus according to one embodiment.

FIG. 2 is an enlarged view of area Q1 in FIG. 1.

FIG. 3 is a view illustrating only a display panel of FIG. 2.

FIG. 4 is a plan view illustrating a pixel arrangement of a display panel according to

one embodiment.

FIG. 5 is a cross-sectional view along line D-D′ in FIG. 4.

FIG. 6 is a cross-sectional view along line E-E′ in FIG. 4.

FIG. 7 is a cross-sectional view of a touch part of FIG. 5 taken at a different angle.

FIG. 8 is a schematic view illustrating a state in which a microlens condenses external light according to the cross-sectional view of FIG. 6.

FIG. 9 is a cross-sectional view along line A-A′ in FIG. 1.

FIG. 10 is a cross-sectional view along line B-B′ in FIG. 3.

FIG. 11 is a cross-sectional view along line C-C′ in FIG. 3.

FIG. 12 is an enlarged plan view illustrating a periphery of a pixel of a display apparatus according to another embodiment.

FIG. 13 is a cross-sectional view along line F-F′ in FIG. 12.

FIG. 14 is an enlarged plan view illustrating a periphery of a pixel of a display apparatus according to still another embodiment.

FIG. 15 is a cross-sectional view along line F-F′ in FIG. 14.

FIG. 16 is an enlarged plan view illustrating a periphery of a pixel of a display apparatus according to yet another embodiment.

FIG. 17 is a cross-sectional view along line H-H′ in FIG. 16.

FIG. 18 is a plan view of a display apparatus according to yet another embodiment.

FIG. 19 is an enlarged view of area Q2 in FIG. 18.

FIG. 20 is a cross-sectional view along line K-K′in FIG. 19.

FIG. 21 is a plan view illustrating arrangement of a pixel of the display apparatus according to yet another embodiment.

### DETAILED DESCRIPTION

Hereinafter, embodiments will be described with reference to the accompanying drawings. In the specification, when a first component (or an area, a layer, a portion, etc.) is described as “on,” “connected,” or “coupled to” a second component, it means that the first component may be directly connected/coupled to the second component or a third component may be disposed therebetween.

To elaborate, as used herein, the term “connected” is intended to have the broadest possible meaning. Specifically, the phrase “A is connected to B” encompasses both a direct connection-where no intervening components or elements are present-and an indirect connection, where one or more intermediate components or elements exist between A and B. In other words, “A is connected to B” includes both direct physical or electrical coupling and indirect coupling through one or more intervening components. Unless explicitly stated otherwise, these terms do not require direct physical or electrical contact. The term “coupled” and “in contact” should be interpreted in the same manner.

The same reference numerals indicate the same components. In addition, for some embodiments in the drawings, thicknesses, proportions, and dimensions of components are exaggerated for effective description of technical contents.

The shapes, sizes, dimensions (e.g., length, width, height, thickness, radius, diameter, area, etc.), ratios, angles, number of elements, and the like illustrated in the accompanying drawings for describing the embodiments of the present disclosure are merely examples, and the present disclosure is not limited thereto.

A dimension including size and a thickness of each component illustrated in the drawing are illustrated for convenience of description, and the present disclosure is not limited to the size and the thickness of the component illustrated, but it is to be noted that the relative dimensions including the relative size, location, and thickness of the components illustrated in various drawings submitted herewith are part of the present disclosure.

The term “and/or” includes all one or more combinations that may be defined by the associated configurations.

Terms such as first and second may be used to describe various components, but the components are not limited by the terms. The terms are used only for the purpose of distinguishing one component from another. For example, a first component may be referred to as a second component, and similarly, the second component may also be referred to as the first component without departing from the scopes of the embodiments. The singular includes the plural unless the context clearly dictates otherwise.

Terms such as “under,” “at a lower side,” “above,” and “at an upper side” are used to describe the relationship between the components illustrated in the drawings. The terms are relative concepts and are described with respect to directions marked in the drawings.

It should be understood that term such as “includes” or “has” is intended to specify the presence of features, numbers, steps, operations, components, parts, or a combination thereof described in the specification and does not preclude the presence or addition possibility of one or more other features, numbers, steps, operations, components, parts, or combinations thereof in advance.

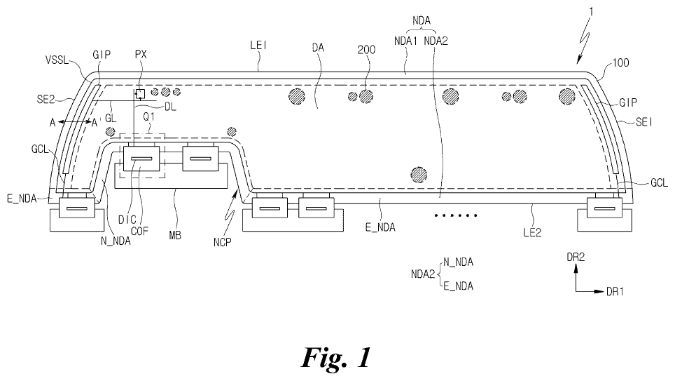

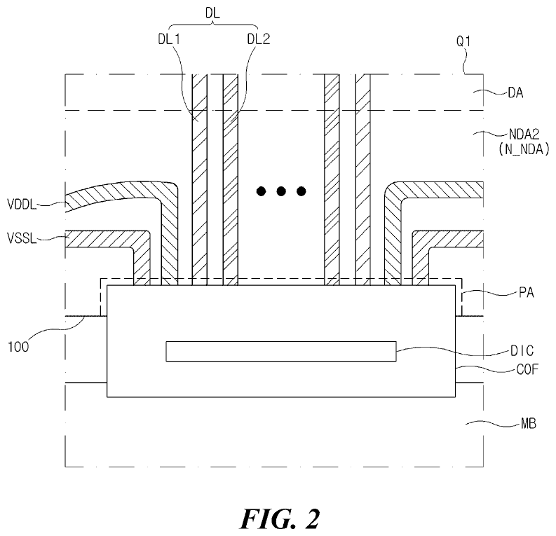

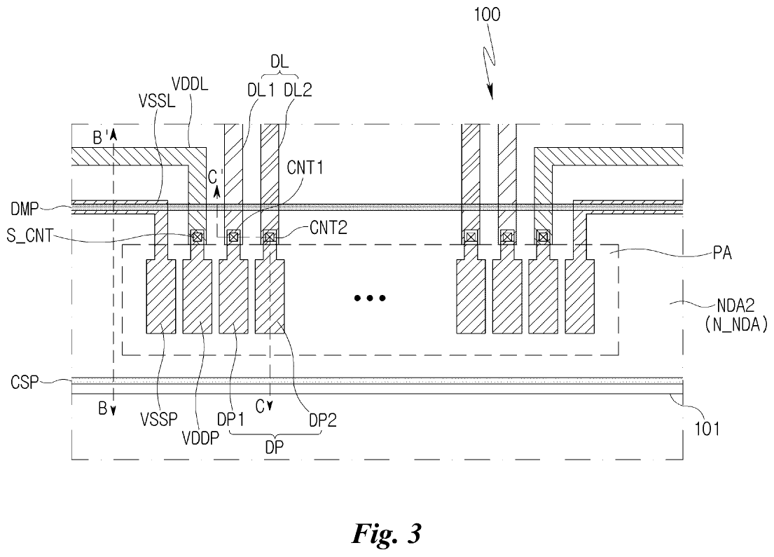

FIG. 1 is a plan view of a display apparatus according to one embodiment. FIG. 2 is an enlarged view of area Q1 in FIG. 1. FIG. 3 is a view illustrating only a display panel of FIG. 2.

FIG. 3 is a view of FIG. 2 from which a flexible film COF, a main board MB, and a drive IC DIC are omitted except for the display panel 100. In FIG. 3, for convenience of description, ratios between components are adjusted.

Referring to FIGS. 1 to 3, a display apparatus 1 may be an apparatus including both a display function for displaying a video and a touch sensing function for sensing touch of a user, but is not limited thereto. For example, the display apparatus 1 may include only one of the display function of displaying an image and the touch sensing function of sensing a user's touch.

The display apparatus 1 may be an electroluminescent display apparatus or a micro light-emitting diode display apparatus that includes a touch sensor. The electroluminescent display apparatus including the touch sensor may be an organic light-emitting diode (OLED) display apparatus, a quantum-dot light-emitting diode display apparatus, or an inorganic light-emitting diode display apparatus.

The display apparatus 1 according to the present embodiment may be a vehicle display apparatus, but is not limited thereto. For example, the description of the display apparatus 1 may be applied without limitation to the type of the apparatus as long as a display apparatus is an apparatus including a display function.

When the display apparatus 1 according to the present embodiment is a vehicle display apparatus, the display apparatus 1 may include a function of manipulating at least some of various functions of a vehicle, a function of displaying various pieces of information about the vehicle, etc.

When the display apparatus 1 according to the present embodiment is a vehicle display apparatus, the display apparatus 1 may be disposed on a dashboard of a vehicle. The display apparatus 1 may be disposed across a driver's seat and a front passenger's seat that are disposed at front seats of a vehicle, but is not limited thereto. Both a driver in the driver's seat and a passenger in the front passenger's seat can use the display apparatus 1.

In some embodiments, a display panel of the display apparatus, or the display apparatus itself, may be integrated into a body of a vehicle. The display apparatus may be fixedly mounted within the vehicle and is not designed for routine removal under normal operating conditions. However, it may be detached or replaced when necessary, such as for repair or maintenance.

The vehicle body may include a motor, which may be an internal combustion engine such as one powered by gasoline or diesel, an electric motor powered by a battery or fuel cell, or a hybrid propulsion system combining both combustion and electric drive sources. Accordingly, the term “vehicle” as used herein includes gasoline-powered vehicles, diesel-powered vehicles, electric vehicles, hybrid electric vehicles, plug-in hybrid electric vehicles, and fuel-cell-powered vehicles, among others.

The display apparatus may be implemented as part of a dashboard-integrated display providing both infotainment and vehicle control functions. It may also be applied to a curved or panoramic cockpit display extending across both driver and passenger zones. In some embodiments, the display apparatus may form part of a head-up display system, where the transmissive area enables light sensing and ambient detection without requiring a through-hole in the optical stack. The display apparatus may also be incorporated into rear-seat entertainment systems or smart partition displays, where the microlens and sensor structure supports dual-view content or enables privacy-controlled viewing. In other configurations, the display may be integrated into a smart mirror or center console system that includes a light-receiving sensor positioned beneath the display to detect ambient light, user presence, or facial orientation.

Further, the display apparatus may support a driver monitoring system by allowing infrared or visible light to pass through the transmissive area and reach an underlying light-receiving sensor. This enables functionalities such as eye-tracking, drowsiness detection, or biometric authentication. The structure may also be suitable for a steering wheel-embedded micro display or a foldable in-cabin display, offering mechanical reliability and a slim profile. Additionally, the apparatus may be implemented in exterior vehicle locations, such as in side-view mirror displays, where compact integration, high transparency, and light-sensing capabilities are advantageous.

The display apparatus 1 may include a display panel 100. The display panel 100 may include a display area DA and a non-display area NDA.

The display area DA may be an area in which light is emitted to the outside to display a screen. The display area DA may further include a function of sensing a user's touch. In this case, the display area DA may correspond to a touch sensing area, but is not limited thereto.

The display area DA may correspond to the shape of the display panel 100, but is not limited thereto.

A plurality of pixels PX may be disposed in the display area DA. The plurality of pixels PX may be repeatedly disposed in a first direction DR1 and a second direction DR2.

The non-display area NDA may be an area in which light is not emitted to the outside so as not to display a screen. The non-display area NDA may be located around the display area DA. The non-display area NDA may surround the display area DA, but the embodiments of the present specification are not limited thereto. A bezel area of the display apparatus 1 may be defined by the non-display area NDA, but the embodiments of the present specification are not limited thereto.

The display panel 100 may be a rigid display panel, but is not limited thereto. The display panel 100 may be a flexible display panel of which shape may be deformed, such as a foldable, bendable, rollable, or stretchable display panel.

The display panel 100 may include a first long edge LE1, a second long edge LE2, a first short edge SE1, and a second short edge SE2 that form an edge of the display panel 100.

The first long edge LE1 and the second long edge LE2 may extend in a first direction DR1, and the first short edge SE1 and the second short edge SE2 may extend in a direction between the first direction DR1 and a second direction DR2. The first long edge LE1 and the second long edge LE2 may have both ends connected through the first short edge SE1 and the second short edge SE2.

The first long edge LE1 may be disposed at one side of the second long edge LE2 in the second direction DR2. The first long edge LE1 and the second long edge LE2 may extend in parallel, but are not limited thereto.

A length of the first long edge LE1 may be shorter than a length of the second long edge LE2. Accordingly, the first short edge SE1 and the second short edge SE2 may extend in an intersecting direction, but are not limited thereto.

The first direction DR1 and the second direction DR2 may be directions intersecting each other. The first direction DR1 and the second direction DR2 may be orthogonal, but are not limited thereto. The first direction DR1 and the second direction DR2 are provided to clarify the description of the disclosure, the first direction DR1 and the second direction DR2 are relative, and the embodiments of the present specification are not limited thereto.

In a plan view, the first long edge LE1 may be disposed above the display area DA, and the second long edge LE2 may be disposed under the display area DA.

In a plan view, the first short edge SE1 may be disposed at the right side of the display area DA, and the second short edge SE2 may be disposed at the left side of the display area DA.

The display panel 100 may include a curved notch NCP. The notch NCP may be formed at the second long edge LE2, but is not limited thereto. That is, the second long edge LE2 may entirely extend in the first direction DR1, but may include the notch NCP that is curved toward the first long edge LE1.

Since the notch NCP is disposed, components, such as a handle of a driver's seat, may be disposed on the corresponding portion to maximize the display area DA capable of displaying the screen, thereby improving a user's convenience and improving aesthetic feeling.

The non-display area NDA may include a first non-display area NDA1 disposed along the first long edge LE1, the first short edge SE1, and the second short edge SE2, and a second non-display area NDA2 disposed along the second long edge LE2. The second non-display area NDA2 may be disposed along the second long edge LE2 including the curved notch NCP.

The first non-display area NDA1 may be disposed at one side and the other side of the display area DA in the first direction DR1 and disposed at one side of the display area DA in the second direction DR2.

The second non-display area NDA2 may include a notch non-display area N_NDA disposed around the notch NCP, and an extension non-display area E_NDA disposed around the notch non-display area N_NDA.

The extension non-display area E_NDA may extend from the notch non-display area N_NDA in the first direction DR1. The extension non-display area E_NDA may be disposed between the notch non-display area N_NDA and the first non-display area NDA1. The extension non-display area E_NDA may connect the notch non-display area N_NDA to the first non-display area NDA1.

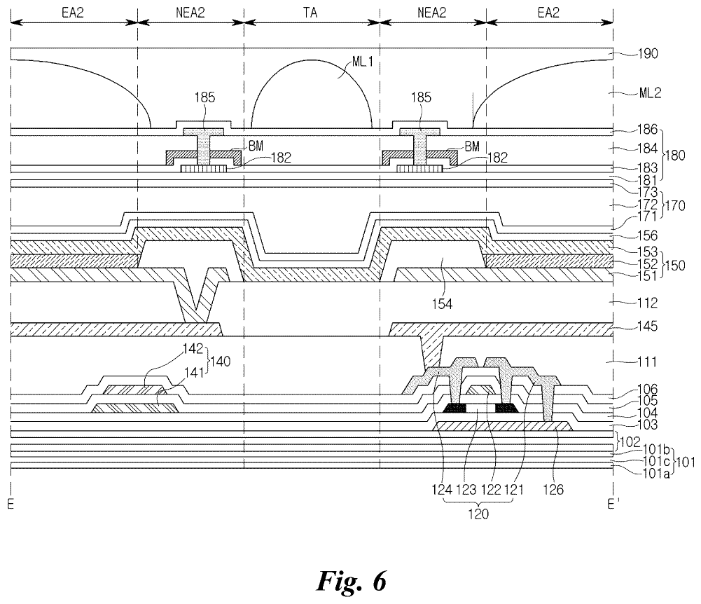

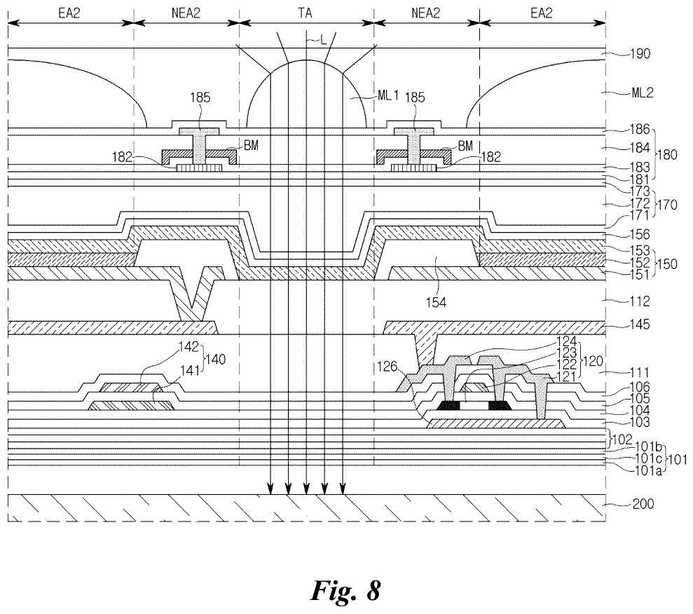

The display apparatus 1 may further include a light-receiving sensor 200. The light-receiving sensor 200 may be disposed on a lower portion of the display panel 100. The light-receiving sensor 200 may be provided as a plurality of light-receiving sensors, but is not limited thereto, and may be provided as a single light-receiving sensor. At least a part of the light-receiving sensor 200 may be disposed to overlap a transmissive area TA (see FIGS. 6 and 8). In some embodiments, the light-receiving sensor 200 is configured to detect at least one of ambient light, infrared light, or biometric characteristics.

In a top view of the display panel 100, the light-receiving sensor 200 may not be visible because it is covered by the display panel 100. However, the light-receiving sensor 200 is illustrated in FIG. 1 in order to describe the size, location, number, etc., of the light-receiving sensor 200 that may be disposed.

The light-receiving sensor 200 may be disposed in the display area DA of the display panel 100. However, the embodiments of the present specification are not limited thereto, and the light-receiving sensor 200 may be disposed in the non-display area NDA or disposed across the display area DA and the non-display area NDA.

The light-receiving sensor 200 may be disposed at various locations in the display area DA. For example, the light-receiving sensor 200 may be disposed along a periphery of the non-display area NDA in the display area DA. However, the embodiments of the present specification are not limited thereto, and the light-receiving sensor 200 may be disposed inside the display area DA away from the non-display area NDA and disposed at various locations according to a design and purpose.

The light-receiving sensor 200 may sense external light incident after passing through the display panel 100. The light-receiving sensor 200 may sense external light that is incident on the display apparatus 1, passes through the display panel 100, and reaches the light-receiving sensor 200.

The light-receiving sensor 200 may perform various operations by sensing external light that reaches the light-receiving sensor 200 after passing through the display panel 100. For example, the light-receiving sensor 200 may perform a function of sensing external light to adjust the illuminance of the display panel 100, detecting infrared rays (IR) among the external light to recognize a driver and a passenger, detecting infrared rays (IR) among the external light to recognize an iris of the driver or the passenger, etc.

However, functions of the light-receiving sensor 200 are not limited thereto, and other functions that are possible by sensing externally incident light may also be performed.

The display apparatus 1 may further include a pad area PA, a gate driving unit GIP, a main board MB, a flexible film COF, a drive IC DIC, a gate line GL, a gate control line GCL, a data line DL, a low-potential voltage line VSSL, and a high-potential voltage line VDDL.

The pad area PA may overlap the flexible film COF. The pad area PA may be attached to the flexible film COF. That is, the display panel 100 and the flexible film COF may be attached through the pad area PA.

The pad area PA may be disposed in the non-display area NDA. The pad area PA may be disposed in the non-display area NDA. The pad area PA may be disposed in the second non-display area NDA2. The pad area PA may be disposed in each of the notch non-display area N_NDA and the extension non-display area E_NDA.

The pad area PA may include a plurality of pads. The pad area PA may include a low-potential voltage pad VSSP, a high-potential voltage pad VDDP, a first data pad DP1, and a second data pad DP2. The low-potential voltage pad VSSP, the high-potential voltage pad VDDP, the first data pad DP1, and the second data pad DP2 may be disposed in the pad area PA.

However, the embodiments of the present specification are not limited thereto, and the pad area PA disposed in an area that overlaps the flexible films COFs disposed at both ends among the flexible films COFs disposed along the non-display area NDA may further include a gate control pad (not illustrated).

The gate driving unit GIP may be disposed in the non-display area NDA. The gate driving unit GIP may be disposed at at least one of one side and the other side of the display area DA in the first direction DR1, but is not limited thereto. In a plan view, the gate driving unit GIP may be disposed at the left side and the other side of the display area DA.

The gate driving unit GIP may include a plurality of transistors. Transistors disposed in the gate driving unit GIP may be connected to the pixels PX through the gate lines GL. The gate driving unit GIP may apply a gate signal to each pixel PX through the gate line GL.

The gate driving unit GIP may receive a gate control signal from the drive IC DIC through the gate control line GCL. The gate driving unit GIP may generate a scan signal and a light-emitting signal (or a light-emitting control signal) based on the gate control signal.

The gate driving unit GIP may include a scan driver and an light-emitting signal driver. The scan driver may generate a scan signal in a row-sequential manner and supply the scan signal to the scan lines in order to drive one or more scan lines connected to each pixel PX row. The light-emitting signal driver may generate an light-emitting signal in a row-sequential manner and supply the light-emitting signal to light-emitting signal lines in order to drive one or more light-emitting signal lines connected to each pixel PX row.

The main board MB may be connected to the display panel 100 through the flexible film COF. The main board MB may be electrically connected to the pixel PX of the display area DA through the flexible film COF. The main board MB may be electrically connected to the flexible film COF. The main board MB and the flexible film COF may be electrically connected through the plurality of pads VSSP, VDDP, and DP.

The main board MB may have various types of components for supplying various signals, such as a gate control signal, a driving signal, a data signal, etc., to the drive IC DIC. The main board MB may be a printed circuit board, but is not limited thereto.

The main board MB may be connected to the display panel 100 through the flexible film COF in the second non-display area NDA2. The main board MB may be provided as a plurality of main boards along the second non-display area NDA2, but is not limited thereto. The number of main boards MB may vary according to a design.

At least one of the main boards MB may be disposed around the notch NCP and connected to the display panel 100 through the flexible film COF in the notch non-display area N_NDA.

The flexible film COF may be connected to the display panel 100 and the main board MB. The flexible film COF may be attached to each of the display panel 100 and the main board MB and electrically connected to each of the display panel 100 and the main board MB. That is, the display panel 100 and the main board MB may be electrically connected through the flexible film COF. The flexible film COF may be provided as a plurality of flexible films, but is not limited thereto.

The flexible film COF may be attached to the display panel 100 in the second non-display area NDA2. The flexible film COF may be repeatedly disposed along the second non-display area NDA2. The flexible film COF may be attached to the display panel 100 across the notch non-display area N_NDA and the extension non-display area E_NDA.

A single main board MB may be electrically connected to the display panel 100 through at least one flexible film COF. For example, the main boards MB disposed at both ends among the plurality of main boards MB disposed along the second non-display area NDA2 may be electrically connected to the display panel 100 through one flexible film COF, and the remaining main boards MB may be electrically connected to the display panel 100 through two flexible films COF.

The flexible film COF may be electrically connected to the pad area PA. Accordingly, the flexible film COF may supply gate control signals, driving signals, power voltages, data voltages, and the like to the plurality of pixels PX and the gate driving unit GIP that are disposed in the display area DA.

The flexible film COF may be a flexible insulating film. The flexible film COF may include, for example, polycarbonate, polyethylene terephthalate, polyimide, polyamide, polyester, polyacrylate, polymethyl methacrylate, etc., but is not limited thereto.

The drive IC DIC may be mounted on the flexible film COF. The drive IC DIC may be disposed by a method of a chip on glass, a chip on film, a tape carrier package, etc., according to a mounting method. In the present disclosure, the drive IC DIC is described as being mounted on the flexible film COF by the chip on film method, but is not limited thereto.

The drive IC DIC may drive the display apparatus 1. The drive IC DIC may process data signals for displaying an image, various driving signals for processing the data signals, etc. The drive IC DIC may include a gate driver IC, a data driver IC, etc.

The gate line GL may be extended from the gate driving unit GIP and connected to the pixel PX. The gate line GL may electrically connect the gate driving unit GIP and the pixel PX. The gate line GL may apply the gate signal from the gate driving unit GIP to each pixel PX.

The gate control line GCL may be disposed in the non-display area NDA. The gate control line GCL may extend from the pad area PA to the gate driving unit GIP and may be electrically connected to the gate driving unit GIP.

The gate control line GCL may apply the gate control signal to the gate driving unit GIP. The gate control signal may be transmitted from the main board MB or the drive IC DIC. The gate control line GCL may electrically connect the gate driving unit GIP to the main board MB or the drive IC DIC.

The gate control line GCL may be electrically connected to the flexible film COF disposed at both ends among the plurality of flexible films COF connected to the display panel 100 along the second non-display area NDA2. The gate control line GCL may be disposed at an outermost edge among a plurality of lines connected to one flexible film COF, but is not limited thereto.

The data line DL may extend from the pad area PA and may be connected to the pixel PX of the display area DA. The data line DL may apply the data signal to each pixel PX. The data signal may be applied from the main board MB or the drive IC DIC. The data line DL may electrically connect the pixel PX to the main board MB or the drive IC DIC.

The data line DL may include a first data line DL1 and a second data line DL2. The data line DL may be connected to the data pads DP1 and DP2. The first data line DL1 may be electrically connected in contact with the first data pad DP1 through a first data contact hole CNT1. The second data line DL2 may be electrically connected in contact with the second data pad DP2 through a second data contact hole CNT2.

The low-potential voltage line VSSL may be disposed in the non-display area NDA to surround the display area DA. The low-potential voltage line VSSL may be disposed in the non-display area NDA with the display area DA and the gate driving unit GIP interposed therebetween. That is, the gate driving unit GIP may be disposed between the display area DA and the low-potential voltage line VSSL.

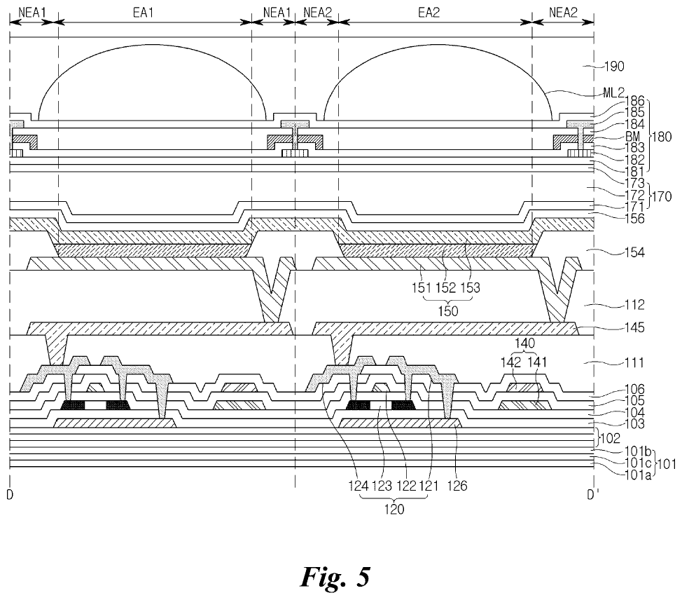

The low-potential voltage line VSSL may apply a low-potential voltage to the pixel PX. The low-potential voltage line VSSL may be electrically connected to a cathode electrode 153 (see FIG. 5) of the pixel PX to apply a low-potential voltage.

The low-potential voltage line VSSL may be connected to the pad area PA. The low-potential voltage line VSSL may be physically connected to the low-potential voltage pad VSSP and electrically connected to the low-potential voltage pad VSSP. The low-potential voltage line VSSL and the low-potential voltage pad VSSP may be formed integrally, but are not limited thereto.

The high-potential voltage line VDDL may be disposed between the display area DA and the low-potential voltage line VSSL. The high-potential voltage line VDDL may apply a high-potential voltage to the pixel PX. The high-potential voltage line VDDL may be electrically connected to an anode electrode 151 (see FIG. 5) of the pixel PX to apply a high-potential voltage.

The high-potential voltage line VDDL may be connected to the pad area PA. The high-potential voltage line VDDL may be physically connected to the high-potential voltage pad VDDP and electrically connected to the high-potential voltage pad VDDP. The high-potential voltage line VDDL may come into contact with the high-potential voltage pad VDDP by a high-potential contact hole S_CNT.

However, the embodiments of the present specification are not limited thereto, and the high-potential voltage line VDDL and the high-potential voltage pad VDDP may be formed integrally. For example, the high-potential voltage line VDDL may be formed of the same material and the same conductive layer as the high-potential voltage pad VDDP, and the high-potential voltage line VDDL and the high-potential voltage pad VDDP are formed together by the same mask process.

The display apparatus 1 may further include a dam part DMP. The dam part DMP may be disposed in the non-display area NDA. The dam part DMP may be disposed to surround the display area DA, but is not limited thereto. At least a part of the dam part DMP may be disposed to overlap the low-potential voltage line VSSL. The dam part DMP may be disposed between the display area DA and the pad area PA in the second non-display area NDA2.

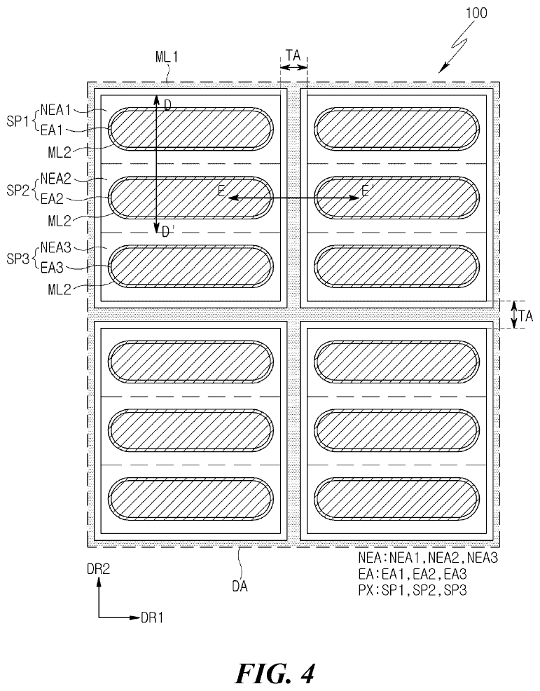

FIG. 4 is a plan view illustrating a pixel arrangement of a display panel according to one embodiment. The plan view of FIG. 4 is an enlarged view illustrating a part of the display area DA in which sub-pixels SP are disposed.

Referring to FIGS. 1 and 4, the display panel 100 may include a plurality of pixels PX, and the plurality of pixels PX may be disposed in the display area DA.

Each of the plurality of pixels PX may include a first sub-pixel SP1, a second sub-pixel SP2, and a third sub-pixel SP3. Each of the first sub-pixel SP1, the second sub-pixel SP2, and the third sub-pixel SP3 may emit light of different colors. For example, the first sub-pixel SP1 may emit red light, the second sub-pixel SP2 may emit green light, and the third sub-pixel SP3 may emit blue light, but the embodiments of the present specification are not limited thereto.

In each pixel PX, the first sub-pixel SP1, the second sub-pixel SP2, and the third sub-pixel SP3 may be disposed sequentially in the second direction DR2. The pixel PX may be repeatedly disposed in the first direction DR1 and the second direction DR2.

Each of the plurality of pixels PX may include a light-emitting area EA and a non-light-emitting area NEA disposed around the light-emitting area EA. Light may be emitted from the light-emitting area EA, and light may not be emitted from the non-light-emitting area NEA. The light-emitting area EA and the non-light-emitting area NEA may be defined by a bank 154 (see FIG. 5).

The light-emitting area EA may include a first light-emitting area EA1 in which light is substantially emitted from the first sub-pixel SP1, a second light-emitting area EA2 in which light is substantially emitted from the second sub-pixel SP2, and a third light-emitting area EA3 in which light is substantially emitted from the third sub-pixel SP3.

The flat surface shape of the light-emitting area EA may have a shape that is longer in the first direction DR1 than in the second direction DR2, but is not limited thereto, and the flat surface shape of the light-emitting area EA may vary according to a design.

The non-light-emitting area NEA may include a first non-light-emitting area NEA1 disposed around the first light-emitting area EA1, a second non-light-emitting area NEA2 disposed around the second light-emitting area EA2, and a third non-light-emitting area NEA3 disposed around the third light-emitting area EA3.

In a plan view, each of the non-light-emitting area NEA (NEA1, NEA2, and NEA3) may surround each of the light-emitting area EA (EA1, EA2, and EA3), but the embodiments of the present specification are not limited thereto.

The first sub-pixel SP1 may include the first light-emitting area EA1 and the first non-light-emitting area NEA1, the second sub-pixel SP2 may include the second light-emitting area EA2 and the second non-light-emitting area NEA2, and the third sub-pixel SP3 may include the third light-emitting area EA3 and the third non-light-emitting area NEA3.

The display panel 100 may further include the transmissive area TA disposed between a plurality of pixels PX. The transmissive area TA may be disposed between the non-light-emitting areas NEA of adjacent pixels PX.

The transmissive area TA may be disposed in the display area DA and disposed in the entire area of the display area DA, but is not limited thereto. For example, a part of the transmissive area TA may be disposed in the non-display area NDA.

A light transmittance of the transmissive area TA may be higher than a light transmittance of an area in which the pixel PX is disposed. That is, the light transmittance of the transmissive area TA may be higher than the light transmittance of the light-emitting area EA and may have higher light transmittance than the non-light-emitting area NEA.

The transmissive area TA may be disposed between the pixels PX and disposed in the first direction DR1 and the second direction DR2. The transmissive area TA between adjacent pixels PX in the first direction DR1 may extend in the second direction DR2, and the transmissive area TA between adjacent pixels PX in the second direction DR2 may extend in the first direction DR1.

A thin film transistor 120 and the bank 154 (see FIG. 5) may not be disposed in the transmissive area TA. For example, the bank 154 is disposed adjacent to the light-emitting part, and the transmissive area TA does not overlap with the bank in a plan view (see FIGS. 5 and 6). A driving transistor and a switching transistor for driving the pixel PX may not be disposed in the transmissive area TA. That is, both the thin film transistor 120, which is a driving transistor, and a switching transistor (not illustrated) may not be disposed in the transmissive area TA.

Since the transmissive area TA is disposed in the entire area of the display area DA, a path for external light to reach the light-receiving sensor 200 can be secured by the transmissive area TA, and thus a through hole for physically removing the respective stacked members for allowing externally incident light to reach the light-receiving sensor 200 can be unnecessary.

Even when the through hole is not disposed, since the transmissive area TA is disposed, the light-receiving sensor 200 can be freely disposed in the display area DA. In addition, the size and shape of the light-receiving sensor 200 can be freely designed. Accordingly, various shapes, sizes, and arrangements of the light-receiving sensor 200 can be possible according to a design or as needed.

Furthermore, since there is no need to change the arrangement and shape of circuit components, etc., of the display panel for arranging the through hole, it is possible to secure the path of light incident on the light-receiving sensor 200 and more efficiently perform the process, thereby reducing production energy.

A first microlens ML1 may be disposed in the transmissive area TA. The first microlens ML1 may condense external light L (see FIG. 8) traveling toward the display panel 100 so that a larger amount of light L (see FIG. 8) passes through the display panel 100.

At least a part of the first microlens ML1 may have a rounded cross section (see FIG. 6). For example, the cross-sectional shape of the first microlens ML1 may semicircular, semi-elliptical, etc. However, the cross-sectional shape of the first microlens ML1 may vary according to a design.

The first microlens ML1 may extend in an extension direction of the transmissive area TA. For example, in the transmissive area TA extending in the first direction DR1, the first microlens ML1 may extend in the first direction DR1, and in the transmissive area TA extending in the second direction DR2, the first microlens ML1 may extend in the second direction DR2.

The first microlens ML1 may be formed integrally in the entire area of the transmissive area TA, but is not limited thereto.

The first microlens ML1 may be disposed in the transmissive area TA, but is not limited thereto, and the first microlens ML1 may be disposed in both the transmissive area TA and a part of the non-light-emitting area NEA around the transmissive area TA.

Since the first microlens ML1 is disposed in the transmissive area TA, the first microlens ML1 may condense the externally incident light L (see FIG. 8) into the transmissive area TA, thereby increasing the amount of light L (see FIG. 8) passing through the display panel 100. Furthermore, a larger amount of light L (see FIG. 8) may be incident on the light-receiving sensor 200 disposed in the transmissive area TA, and the light-receiving sensor 200 may sense the light L (see FIG. 8) more smoothly.

A second microlens ML2 may be disposed in each sub-pixel SP1, SP2, or SP3. The second microlens ML2 may be disposed in each sub-pixel SP1, SP2, or SP3.

The second microlens ML2 may control a traveling direction of the light emitted from the light-emitting area EA. Accordingly, when the display panel 100 is applied to a display apparatus for a vehicle, screens displayed to the driver and the passenger may be separately provided and controlled.

In addition, by arranging the second microlens ML2 in the light-emitting area EA, it is possible to secure the wide viewing angle characteristics, increase luminance, and block light leakage, reflected light, etc., thereby preventing light leakage.

The second microlens ML2 may be disposed in each light-emitting area EA1, EA2, or EA3. The second microlens ML2 may cover each light-emitting area EA1, EA2, or EA3 and may be disposed in each light-emitting area EA1, EA2, or EA3, and a part of the non-light-emitting area NEA1, NEA2, or NEA3 around each light-emitting area EA1, EA2, or EA3. However, the embodiments of the present specification are not limited thereto, and the second microlens ML2 may be disposed only in each light-emitting area EA1, EA2, or EA3.

At least a part of the second microlens ML2 may have a rounded cross section (see FIG. 6). For example, the cross-sectional shape of the second microlens ML2 may semicircular, semi-elliptical, etc. However, the cross-sectional shape of the second microlens ML2 may vary according to a design. In some embodiments, the second microlens ML2 covers at least a portion of both the light-emitting area EA1 and the non-light-emitting area NEA1 in a plan view (see FIG. 5).

A surface of the second microlens ML2 may have a rounded shape. For example, the surface of the second microlens ML2 may have a dome shape, but is not limited thereto.

The second microlens ML2 is illustrated as being disposed in each sub-pixel SP1, SP2, or SP3, but is not limited thereto. For example, according to the design of each sub-pixel SP1, SP2, or SP3, the second microlens ML2 disposed in each sub-pixel SP1, SP2, or SP3 may be provided as two or more microlenses. When the opening (the light-emitting area EA) formed in one sub-pixel SP1, SP2, or SP3 is provided as a plurality of openings, the second microlens ML2 may be disposed in each opening, or a plurality of second microlenses ML2 may be disposed in one opening.

Hereinafter, a cross-sectional structure of the display area DA of the display panel 100 including the pixels PX will be described with reference to FIGS. 5 to 7.

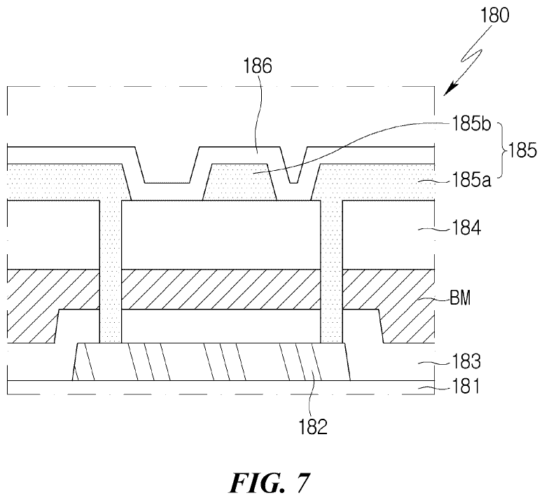

FIG. 5 is a cross-sectional view along line D-D′ in FIG. 4. FIG. 6 is a cross-sectional view along line E-E′ in FIG. 4. FIG. 7 is a cross-sectional view of a touch part of FIG. 5 taken at a different angle. FIG. 8 is a schematic view illustrating a state in which a microlens condenses external light according to the cross-sectional view of FIG. 6.

FIG. 6 does not illustrate the light-receiving sensor 200, but when the light-receiving sensor 200 is disposed, at least a part of the light-receiving sensor 200 may be disposed to overlap the transmissive area TA. Accordingly, externally incident light may pass through the transmissive area TA of the display panel 100 and reach the light-receiving sensor 200.

Referring to FIGS. 4 to 8, the display panel 100 may include a substrate 101, the thin film transistor 120, a storage electrode 140, a light-emitting part 150, an encapsulation part 170, a touch part 180, etc. In addition, the display panel 100 may be disposed on the substrate 101 and may further include a buffer layer 102, a first insulating layer 103, a second insulating layer 104, a third insulating layer 105, a fourth insulating layer 106, a first protective layer 111, a second protective layer 112, and the bank 154, which are disposed between components. However, the embodiments of the present specification are not limited thereto.

As used herein, the term “a light-emitting part 150” broadly refers to a structure that emits light in response to an electrical signal and is responsible for generating image display in the display area. The light-emitting part may include various configurations, such as an organic light-emitting diode (OLED) structure having an anode electrode, one or more organic material layers (e.g., emission layers, transport layers, injection layers), and a cathode electrode. The light-emitting part may also include inorganic light-emitting diodes (e.g., micro LEDs or mini LEDs), quantum-dot emissive structures, or hybrid configurations combining organic and inorganic emissive materials. The specific structure and composition of the light-emitting part may vary depending on the display technology used, and the term is intended to encompass any configuration that performs the function of emitting light within a pixel or subpixel of the display panel.

An area in which each pixel PX is disposed (the light-emitting area EA and the non-light-emitting area NEA) and the transmissive area TA may have different stacking structures.

The substrate 101, the thin film transistor 120, the storage electrode 140, the light-emitting part 150, the encapsulation part 170, and the touch part 180, etc., may be disposed in the area in which each pixel PX is disposed (the light-emitting area EA and the non-light-emitting area NEA), and the buffer layer 102, the first insulating layer 103, the second insulating layer 104, the third insulating layer 105, the fourth insulating layer 106, the first protective layer 111, the second protective layer 112, the bank 154, etc., may be further disposed between components.

Only components with high transmittance may be disposed in the transmissive area TA, and components with low transmittance may not be disposed. For example, in the transmissive area TA, the substrate 101, a part (the cathode electrode 153) of the light-emitting part 150, the encapsulation part 170, the touch part 180, etc., may be disposed, and the buffer layer 102, the first insulating layer 103, the second insulating layer 104, the third insulating layer 105, the fourth insulating layer 106, the first protective layer 111, the second protective layer 112, the bank 154, etc., may be further disposed between components. In the transmissive area TA, the thin film transistor 120, the storage electrode 140, the bank 154, and the remaining parts (the anode electrode 151 and an organic layer 152) of the light-emitting part 150 may not be disposed.

Since only components with high transmittance are disposed in the transmissive area TA, it is possible to increase the light transmittance of the transmissive area TA.

The substrate 101 may provide a space in which various components may be disposed thereon. The substrate 101 may correspond to the flat surface shape of the display panel 100 of FIG. 1. That is, the substrate 101 may include the notch NCP. The substrate 101 may include the display area DA and the non-display area NDA of the display panel 100 in substantially the same manner.

The substrate 101 may include one or more plastic materials, but is not limited thereto, and may include a glass material.

The substrate 101 may be a multi-substrate including a plurality of substrates of a first substrate 101a, a second substrate 101b, and a third substrate 103c each including a plastic material, such as polyimide, but the embodiments of the present specification are not limited thereto. For example, the substrate 101 may be a single substrate formed of a single layer.

The substrate 101 may include a rigid substrate. However, the embodiments of the present specification are not limited thereto, and the substrate 101 may include a flexible substrate.

The buffer layer 102 may be disposed on the substrate 101. The buffer layer 102 can minimize or delay the diffusion of moisture or oxygen penetrating the substrate 101. The buffer layer 102 may be formed by alternately stacking silicon nitride (SiNₓ) and silicon oxide (SiOₓ) at least once, but the embodiments of the present specification are not limited thereto.

The specification describes that the buffer layer 102 is formed as multiple layers formed of three layers, but the number of layers forming the buffer layer 102 is not limited thereto, and the buffer layer 102 may be formed as a single layer.

A light-shielding layer 126 may be disposed on the buffer layer 102. The light-shielding layer 126 can prevent light from being transmitted to a semiconductor layer 123 of the thin film transistor 120. For example, the semiconductor layer 123 may be disposed to overlap the light-shielding layer 126. The light-shielding layer 126 may be formed of a single layer or multiple layers formed of one of molybdenum (Mo), aluminum (Al), chromium (Cr), nickel (Ni), neodymium (Nd), and copper (Cu) or an alloy thereof, but the embodiments of the present specification are not limited thereto.

The light-shielding layer 126 may come into contact with a source electrode 121 of the thin film transistor 120 through a contact hole.

The first insulating layer 103 may be disposed on the light-shielding layer 126. The first insulating layer 103 can prevent a short circuit between a component of the thin film transistor 120 and the light-shielding layer 126. The first insulating layer 103 may be formed of the same material as the buffer layer 102, but the embodiments of the present specification are not limited thereto. For example, the first insulating layer 103 may be formed of an inorganic material, such as silicon nitride (SiNₓ) or silicon oxide (SiOₓ), but the embodiments of the present specification are not limited thereto.

The thin film transistor 120 may be disposed on the first insulating layer 103. The thin film transistor 120 may include the source electrode 121, a gate electrode 122, a semiconductor layer 123, and a drain electrode 124.

The thin film transistor 120 may be disposed in the area (the light-emitting area EA and the non-light-emitting area NEA) in which the pixel PX is disposed and may not be disposed in the transmissive area TA.

The semiconductor layer 123 may be disposed on the first insulating layer 103. The semiconductor layer 123 may include a metal oxide semiconductor, such as indium-gallium-zinc oxide (IGZO), and a silicon-based semiconductor material, such as amorphous silicon or polycrystalline silicon, but the embodiments of the present specification are not limited thereto. The semiconductor layer 123 may include a source area, a drain area, and a channel area between the source area and the drain area.

Since the polycrystalline semiconductor layer has higher mobility than the amorphous semiconductor layer and the oxide semiconductor layer, power consumption can be less, and reliability can be excellent. Accordingly, a driving transistor may be formed of a polycrystalline semiconductor layer, but the embodiments of the present specification are not limited thereto.

The second insulating layer 104 may be disposed on the semiconductor layer 123. The second insulating layer 104 may be formed of the same material as the first insulating layer 103, but the embodiments of the present specification are not limited thereto. The second insulating layer 104 can prevent a short circuit between the semiconductor layer 123 and another component of the thin film transistor 120.

The gate electrode 122 may be disposed on the second insulating layer 104. The gate electrode 122 may be disposed on the second insulating layer 104 to overlap the channel area of the semiconductor layer 123. The gate electrode 122 may be formed of a single layer or multiple layers made of molybdenum (Mo), copper (Cu), titanium (Ti), aluminum (Al), chromium (Cr), gold (Au), nickel (Ni), neodymium (Nd), or a compound thereof, but the embodiments of the present specification are not limited thereto. The gate electrode 122 may be disposed along with the gate line, but the embodiments of the present specification are not limited thereto.

The third insulating layer 105 may be disposed on the gate electrode 122. The third insulating layer 105 may be formed of the same material as the first insulating layer 103 or the second insulating layer 104, but the embodiments of the present specification are not limited thereto.

The storage electrode 140 may be disposed to be spaced apart from the thin film transistor 120. The storage electrode 140 may include a first storage electrode 141 and a second storage electrode 142.

The storage electrode 140 may be disposed in the area (the light-emitting area EA and the non-light-emitting area NEA) in which the pixel PX is disposed and may not be disposed in the transmissive area TA.

The first storage electrode 141 may be formed of the same material as the gate electrode 122 and formed on the same layer, but the embodiments of the present specification are not limited thereto.

The second storage electrode 142 may be disposed on the first storage electrode 141. The second storage electrode 142 may be disposed on the third insulating layer 105, and the third insulating layer 105 between the first storage electrode 141 and the second storage electrode 142 may be used as a dielectric to generate a capacitance. The second storage electrode 142 may be formed of the same material as the first storage electrode 141, but the embodiments of the present specification are not limited thereto.

The fourth insulating layer 106 may be disposed on the second storage electrode 142. The fourth insulating layer 106 may be formed of the same material as the first insulating layer 103, the second insulating layer 104, or the third insulating layer 105, but the embodiments of the present specification are not limited thereto.

The source electrode 121 and the drain electrode 124 may be disposed on the fourth insulating layer 106.

The source electrode 121 and the drain electrode 124 may be electrically connected to the semiconductor layer 123 through contact holes. At least one of the source electrode 121 and the drain electrode 124 may come into contact with the light-shielding layer 126 through a contact hole. For example, the source electrode 121 may come into contact with the light-shielding layer 126 through the contact hole.

The source electrode 121 and the drain electrode 124 may be formed of a metallic material. For example, the source electrode 121 and the drain electrode 124 may be formed of a single layer or multiple layers made of one of molybdenum (Mo), aluminum (Al), chromium (Cr), gold (Au), titanium (Ti), nickel (Ni), neodymium (Nd), and copper (Cu) or an alloy thereof, but the embodiments of the present specification are not limited thereto.

The source electrode 121 and the drain electrode 124 may be disposed along with the data line. For example, the data line may be formed of the same material as the source electrode 121 and the drain electrode 124 and formed on the same layer, but the embodiments of the present specification are not limited thereto.

The thin film transistor 120 may be a driving transistor, and although not illustrated, the display panel 100 may further include a switching transistor, but the embodiments of the present specification are not limited thereto.

The first protective layer 111 may be disposed on the source electrode 121 and the drain electrode 124.

The first protective layer 111 may planarize an upper portion of the thin film transistor 120 and protect the thin film transistor 120. The first protective layer 111 may be formed of an organic material. For example, the first protective layer 111 may be formed of an organic material including an acrylic resin, an epoxy resin, a phenolic resin, a polyamide resin, or a polyimide resin, but the embodiments of the present specification are not limited thereto.

The second protective layer 112 may be disposed on the first protective layer 111. The second protective layer 112 may be formed of the same material as the first protective layer 111, but the embodiments of the present specification are not limited thereto.

A connection electrode 145 may be disposed between the first protective layer 111 and the second protective layer 112.

The connection electrode 145 may be disposed in the area (the light-emitting area EA and the non-light-emitting area NEA) in which the pixel PX is disposed and may not be disposed in the transmissive area TA.

The connection electrode 145 may electrically connect the thin film transistor 120 to the light-emitting part 150. The connection electrode 145 may be formed of the same material as the source electrode 121 and the drain electrode 124, but the embodiments of the present specification are not limited thereto.

The connection electrode 145 may come into contact with the drain electrode 124 through the contact hole formed in the first protective layer 111 and may be electrically connected to the drain electrode 124.

The connection electrode 145 may be formed of a single layer or multiple layers made of any one of molybdenum (Mo), aluminum (Al), chromium (Cr), gold (Au), titanium (Ti), nickel (Ni), neodymium (Nd), and copper (Cu) or an alloy thereof, but the embodiments of the present specification are not limited thereto.

The light-emitting part 150 may be disposed on the second protective layer 112. The light-emitting part 150 may include the anode electrode 151, the organic layer 152, and the cathode electrode 153.

The anode electrode 151 and the organic layer 152 may be disposed in the area in which the pixel PX is disposed (the light-emitting area EA and the non-light-emitting area NEA) and may not be disposed in the transmissive area TA. The cathode electrode 153 may be disposed in the area (the light-emitting area EA and the non-light-emitting area NEA) in which the pixel PX is disposed and may not be disposed in the transmissive area TA. The cathode electrode 153 may be disposed in the entire area of the display area DA.

The anode electrode 151 may be disposed on the second protective layer 112. The anode electrode 151 may be electrically connected to the thin film transistor 120 through a contact hole formed in the second protective layer 112.

The anode electrode 151 may be a reflective electrode that reflects light, but the embodiments of the present specification are not limited thereto. The anode electrode 151 may include a metallic material with high reflectivity, such as a stacking structure (Ti/Al/Ti) of aluminum (Al) and titanium (Ti), a stacking structure (ITO/AI/ITO) of aluminum (Al) and indium tin oxide (ITO), or an APC alloy and may be formed of a single layer or multiple layers, but the embodiments of the present specification are not limited thereto.

The organic layer 152 may be disposed on the anode electrode 151. The organic layer 152 may include one or more light-emitting structures (or light-emitting elements or elements) stacked on the anode electrode 151 in the order or reverse order of a hole transfer layer and an electron transfer layer. For example, the hole transfer layer may include a hole transporting layer, a hole injecting layer, an electron blocking layer, a p-type charge generation layer, etc., but the embodiments of the present specification are not limited thereto. For example, the electron transfer layer may include an electron transporting layer, an electron injecting layer, a hole blocking layer, an n-type charge generation layer, etc., but the embodiments of the present specification are not limited thereto.

The organic layer 152 may be an organic light-emitting layer, an inorganic light-emitting layer, a quantum dot light-emitting layer, a micro light-emitting diode, a micro mini light-emitting diode, etc., but the embodiments of the present specification area not limited thereto. For example, the organic layer 152 of the display panel 100 according to one embodiment of the present specification may include an organic light-emitting layer. The organic layer 152 may be a white light-emitting layer, but the embodiments of the present specification are not limited thereto. The organic layer 152 may be a white light-emitting layer, but the embodiments of the present specification are not limited thereto.

The cathode electrode 153 may be disposed on the organic layer 152. The cathode electrode 153 may be a transparent electrode that transmits light, but the embodiments of the present specification are not limited thereto. For example, the cathode electrode 153 may include a transparent conductive material, such as indium tin oxide (ITO) or indium zinc oxide (IZO), or a metal that transmits visible light, but the embodiments of the present specification are not limited thereto.

A capping layer 156 may be further disposed on the cathode electrode 153. The capping layer 156 can minimize damage to the cathode electrode 153 of the light-emitting element EL and the organic layers 152 located below the cathode electrode 153 from an external light source. The capping layer 156 may be formed of an organic or inorganic film.

The capping layer 156 may be disposed using a material, such as LiF or the like, as an inorganic film and may further include an organic film, but the embodiments of the present specification are not limited thereto. For example, the capping layer 156 may be formed of the stacking structure of an organic film and an inorganic film, and a thickness of the organic film may differ from a thickness of the inorganic film. In this case, the thickness of the organic film may be greater than the thickness of the inorganic film. As another example, the capping layer 156 may be formed of two or more layers by stacking materials having different refractive indexes. Accordingly, it is possible to increase the light efficiency of the display panel 100.

A bank 154 may be disposed to expose the anode electrode 151. The bank 154 may define the opening (or the light-emitting area EA) of the sub-pixel SP1, SP2, or SP3 and may be disposed to cover an edge of the anode electrode 151. The bank 154 may be disposed at boundaries between adjacent sub-pixels SP1, SP2, and SP3.

The organic layer 152 may be disposed in the opening of the sub-pixel SP. That is, the organic layer 152 may be disposed on the anode electrode 151 exposed by the bank 154.

The bank 154 may be formed of a material containing black pigment, or an organic material, such as a benzocyclobutene resin, a polyimide resin, an acrylic resin, a photosensitive polymer, etc., but the embodiments of the present specification are not limited thereto. When the bank 154 is formed of a material containing black pigment or black dye, the bank 154 may be an opaque bank. When the bank 154 is formed of a material containing black pigment or black dye, it is possible to shield external light or light reflected from the outside, thereby further increasing the luminance of the display apparatus.

The bank 154 may be disposed in the area (the light-emitting area EA and the non-light-emitting area NEA) in which the pixel PX is disposed and may not be disposed in the transmissive area TA.

A spacer (not illustrated) may be further disposed on the bank 154. The spacer (not illustrated) may be formed of the same material as the bank 154, but the embodiments of the present specification are not limited thereto. The spacer (not illustrated) can prevent sagging of a mask during a mask process, thereby suppressing or preventing defects, such as imprinting, scratching, or the like, on the display panel 100.

The encapsulation part 170 may be disposed on the bank 154 or the light-emitting part 150. The encapsulation part 170 may include one or more insulating layers. For example, the encapsulation part 170 may include a first inorganic encapsulation layer 171, an organic encapsulation layer 172 formed on the first inorganic encapsulation layer 171, and a second inorganic encapsulation layer 173 formed on the organic encapsulation layer 172. The encapsulation part 170 may include one or more inorganic layers and one or more organic layers. For example, the first inorganic encapsulation layer 171 and the second inorganic encapsulation layer 173 may include an inorganic material, and the organic encapsulation layer 172 may include an organic material, but the embodiments of the present specification are not limited thereto.

Even when the first inorganic encapsulation layer 171 and the second inorganic encapsulation layer 173 may be disposed to extend to an end of the non-display area NDA, the organic encapsulation layer 172 may be ended inside the dam part DMP. That is, the organic encapsulation layer 172 may be disposed inside an area surrounded by the dam part DMP without extending beyond the dam part DMP.

The touch part 180 may be disposed on the encapsulation part 170. The touch part 180 may include a touch buffer layer 181, a first touch electrode 182, a first touch insulating layer 183, a black matrix BM, a second touch insulating layer 184, a second touch electrode 185, and a third touch insulating layer 186.

The touch buffer layer 181 may be disposed on the encapsulation part 170. For example, the touch buffer layer 181 may be disposed on the second inorganic encapsulation layer 173. The touch buffer layer 181 may be formed of the same material as the buffer layer 102, but the embodiments of the present specification are not limited thereto.

The first touch electrode 182 may be disposed on the touch buffer layer 181.

The first touch insulating layer 183 may be disposed on the first touch electrode 182. The first touch insulating layer 183 may be formed of silicon oxide (SiOₓ), silicon nitride (SiNₓ), or multiple layers thereof, but the embodiments of the present specification are not limited thereto.

The black matrix BM may be disposed on the first touch insulating layer 183. The black matrix BM may include materials capable of absorbing light. The black matrix BM may include a black pigment or dye, but is not limited thereto. The black matrix BM can prevent a light leakage defect, etc., that may occur between the sub-pixels SP.

The second touch insulating layer 184 may be disposed on the black matrix BM. The second touch insulating layer 184 may include an organic insulation material. For example, the second touch insulating layer 184 may be formed of photo acryl, benzocyclobutene (BCB), polyimide (PI), or polyamide (PA), but is not limited thereto.

The second touch electrode 185 may be disposed on the second touch insulation layer 184. The second touch electrode 185 may include a 1a touch electrode 185a extending in the first direction DR1 and a 1b touch electrode 185b extending in the second direction DR2 different from the first direction.

The first touch electrode 182 may be electrically connected to a 2a touch electrode 185a through a contact hole formed in the insulating layer 184. For example, the 2a touch electrode 185a and the first touch electrode 182 may extend in the first direction DR1.

The first touch electrode 182 and the second touch electrode 185 may include a metallic material. For example, the sensor electrode 185 and the bridge electrode 182 may be formed of titanium (Ti), nickel (Ni), aluminum (Al), or an alloy thereof and formed of a triple layer, such as titanium (Ti)/aluminum (Al)/titanium (Ti), but the embodiments of the present specification are not limited thereto.

One of the first touch electrode 182 and the second touch electrode 185 may include a function of detecting touch, and the other may include a function of driving touch, but the embodiments of the present specification are not limited thereto.

The third touch insulating layer 186 may be disposed on the second touch electrode 185. The third touch insulating layer 186 may be formed of the same material as the first touch insulating layer 183, but is not limited thereto.

The microlens ML1 and ML2 may be disposed on the third touch insulating layer 186. The first microlens ML1 may be disposed in the transmissive area TA, and the second microlens ML2 may be disposed in each sub-pixel SP1, SP2, or SP3. The first microlens ML1 and the second microlens ML2 may be disposed on the same layer, but are not limited thereto.

Since the first microlens ML1 is disposed in the transmissive area TA, the first microlens ML1 may condense externally incident light L into the transmissive area TA, thereby increasing the amount of light L passing through the display panel 100. Furthermore, a larger amount of light L may be incident on the light-receiving sensor 200 disposed in the transmissive area TA, and the light-receiving sensor 200 may sense the light L more smoothly.

A lens protective film 190 may be disposed on the microlenses ML1 and ML2. The lens protective film 190 may include an organic insulation material, but is not limited thereto. The lens protective film 190 may protect the microlenses ML1 and ML2 by covering the microlenses ML1 and ML2.

A refractive index of the lens protective film 190 may be smaller than refractive indexes of the microlenses ML1 and ML2. Accordingly, due to a difference in refractive index between the microlenses ML1 and ML2 and the lens protective layer 190, light that has passed through the microlenses ML1 and ML2 can be prevented from being reflected toward the substrate 101.

Hereinafter, a cross-sectional structure of the non-display area NDA of the display apparatus 1 will be described. The same content as that described in the cross-sectional structure of the display area DA will be briefly described or omitted.

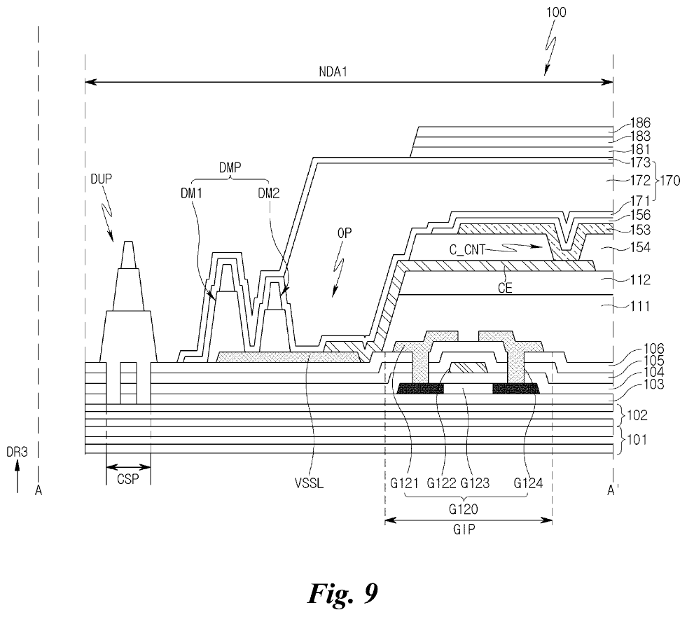

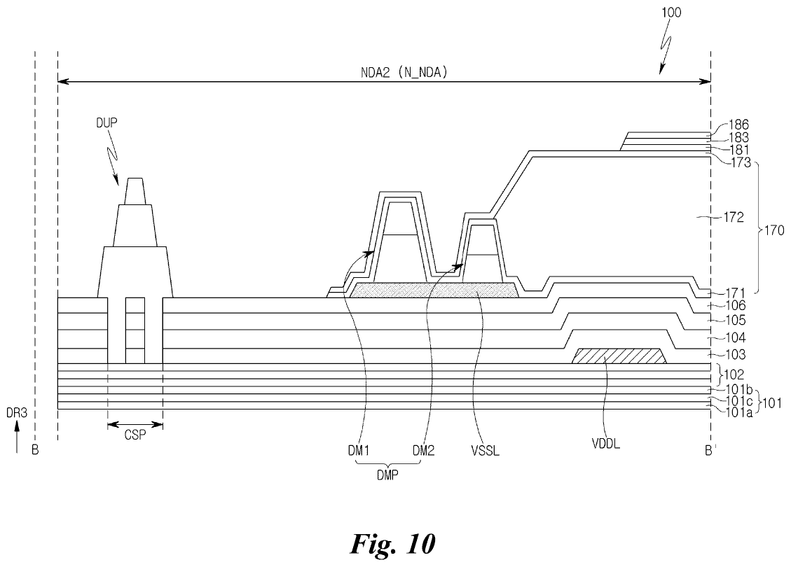

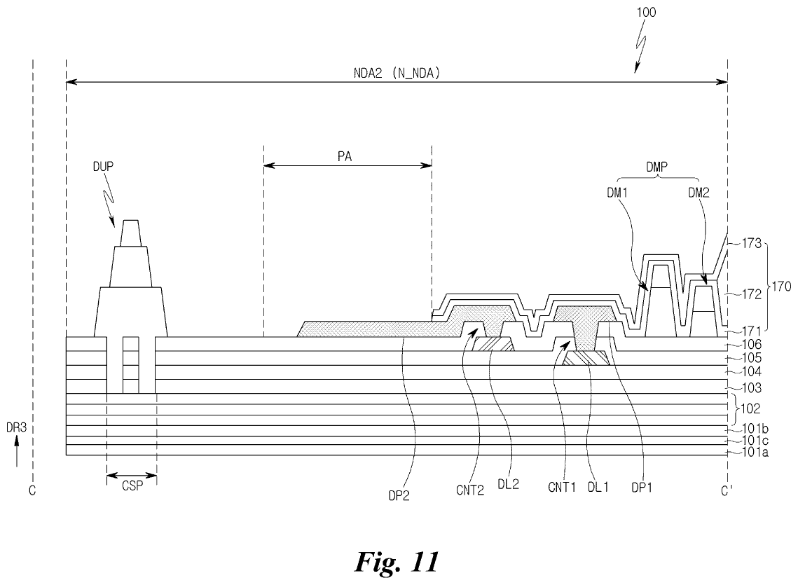

FIG. 9 is a cross-sectional view along line A-A′ in FIG. 1. FIG. 10 is a cross-sectional view along line B-B′in FIG. 3. FIG. 11 is a cross-sectional view along line C-C′ in FIG. 3.

FIG. 9 illustrates a cross-sectional structure of the first non-display area NDA1. FIGS. 10 and 11 illustrate cross-sectional structures of the second non-display area NDA2. FIGS. 10 and 11 illustrate cross sections of the notch non-display area N_NDA of the second non-display area NDA2, but the descriptions thereof may also be applied to the extension non-display area E_NDA in the substantially the same manner.

Referring further to FIGS. 1, 3, 5, and 9 to 11, the display panel 100 may further include the gate control transistor G120, the low-potential voltage line VSSL, the dam part DMP, the plurality of pads VSSP, VDDP, and DP disposed in the pad area PA, the data line DL (DL1 and DL2), and a crack prevention pattern CSP, which are disposed in the non-display area NDA.

The gate control transistor G120 may have substantially the same configuration as the thin film transistor 120 of the sub-pixel SP and may be formed together by the same process as the thin film transistor 120 of the sub-pixel SP, but is not limited thereto.

The gate control transistor G120 may include a control source electrode G121, a control gate electrode G122, a control semiconductor layer G123, and a control drain electrode G124.

A light-shielding layer (not illustrated) may be further disposed under the gate control transistor G120. One of the control source electrode G121 and the control drain electrode G124 may be electrically connected in contact with the light-shielding layer (not illustrated), but is not limited thereto.

The low-potential voltage line VSSL may be disposed on the fourth insulating layer 106. The low-potential voltage line VSSL may be formed of the same metal layer as the source electrode 121 and the drain electrode 124 of the thin film transistor 120, but is not limited thereto.

The display panel 100 may further include a low-potential connection electrode CE. The low-potential connection electrode CE may connect the low-potential voltage line VSSL to the cathode electrode 153.

The low-potential connection electrode CE may be disposed on the second protective layer 112. The bank 154 may be disposed on the low-potential connection electrode CE. The low-potential connection electrode CE may be disposed on the same layer as the anode electrode 151 and may include the same material as the anode electrode 151, and the low-potential connection electrode CE and the anode electrode 151 may be formed together using one mask by the same process, but the embodiments of the present specification are not limited thereto.

The display panel 100 may further include an exposed part OP. The exposed part OP may expose at least a part of the low-potential voltage line VSSL by recessing the first protective layer 111 and the second protective layer 112.

The exposed part OP may be defined by the first protective layer 111 and the second protective layer 112. The exposed part OP may be defined by a side surface of the first protective layer 111, a side surface of the second protective layer 112, and a side surface of a second dam DM2.

The low-potential connection electrode CE may be electrically connected in contact with the low-potential voltage line VSSL exposed in the exposed part OP. At least a part of the low-potential connection electrode CE may be disposed on the second protective layer 112 and may extend from the second protective layer 112 toward the low-potential voltage line VSSL.

The low-potential connection electrode CE may be further disposed on the side surface of the first protective layer 111 that defines the exposed part OP and the side surface of the second protective layer 112 and may be further disposed on the fourth insulating layer 106 and the low-potential voltage line VSSL that are exposed by the exposed part OP. Accordingly, the low-potential connection electrode CE may come into contact with the low-potential voltage line VSSL.

The low-potential connection electrode CE may be electrically connected to the cathode electrode 153. The low-potential connection electrode CE and the cathode electrode 153 may be electrically connected in contact with each other through a low-potential contact hole C_CNT in an overlapping area. The low-potential contact hole C_CNT may be defined by passing through the bank 154 in the area in which the low-potential connection electrode CE and the cathode electrode 153 overlap each other and may expose the low-potential connection electrode CE.

The dam part DMP may include a first dam DM1 and a second dam DM2. The first dam DM1 and the second dam DM2 may overlap a first low-potential voltage line VSSL1 or a second low-potential voltage line VSSL2.

In the second non-display area NDA2, the first dam DM1 and the second dam DM2 may overlap the first low-potential voltage line VSSL1. In the first non-display area NDA1, the first dam DM1 and the second dam DM2 may overlap the second low-potential voltage line VSSL2.

The first dam DM1 may be disposed outside the second dam DM2, but is not limited thereto.

The first dam DM1 may be formed in a multilayered structure. Each layer of the first dam DM1 may include the same material as the second protective layer 112 and the bank 154, and each layer of the first dam DM1, the second protective layer 112, and the bank 154 may be formed together using one mask by the same process, but the embodiments of the present specification are not limited thereto.

The second dam DM2 may be formed in a multilayered structure. Each layer of the second dam DM2 may include the same material as the bank 154 and the spacer (not illustrated), and each layer of the second dam DM2, the bank 154, and the spacer (not illustrated) may be formed together using one mask by the same process, but the embodiments of the present specification are not limited thereto.

The crack prevention pattern CSP may be disposed at an outermost edge of the non-display area NDA. The crack prevention pattern CSP may be defined by recessing at least one of the inorganic films disposed on the substrate 101.

For example, the crack protection pattern CSP may be defined by recessing the first insulating layer 103, the second insulating layer 104, the third insulating layer 105, and the fourth insulating layer 106, but is not limited thereto.

A crack dummy pattern DUP may be further disposed on the crack protection pattern CSP. The crack dummy pattern DUP may fill the recessed crack protection pattern CSP. The crack dummy pattern DUP may be formed of multiple layers. For example, the crack dummy pattern DUP may be formed of three layers. Layers of the crack dummy pattern DUP may include the same material as the first protective layer 111, the second protective layer 112, and the bank 154.

The high-potential voltage line VDDL may be disposed on the buffer layer 102 and covered by the first insulating layer 103. The high-potential voltage line VDDL may include the same material as the light-shielding layer 126, and the high-potential voltage line VDDL and the light-shielding layer 126 may be formed together using one mask by the same process, but the embodiments of the present specification are not limited thereto.

Although not illustrated, the high-potential voltage pad VDDP may be disposed on the same layer as the source electrode 121 and the drain electrode 124, may include the same material as the source electrode 121 and the drain electrode 124, and may be formed together using one mask by the same process as the source electrode 121 and the drain electrode 124, but is not limited thereto.

In this case, the high-potential voltage pad VDDP may be electrically connected in contact with the high-potential voltage line VDDL through the high-potential contact hole S_CNT that exposes the high-potential voltage line VDDL.

However, the embodiments of the present specification are not limited thereto, and the high-potential voltage line VDDL may be disposed on the same layer as the source electrode 121 and the drain electrode 124 and may include the same material as the source electrode 121 and the drain electrode 124, and the high-potential voltage line VDDL, the source electrode 121, and the drain electrode 124 may be formed together using one mask by the same process.

The first data pad DP1 and the second data pad DP2 may be disposed on the fourth insulating layer 106. The first data pad DP1 and the second data pad DP2 may be disposed on the same layer as the source electrode 121 and the drain electrode 124, may include the same material as the source electrode 121 and the drain electrode 124, and may be formed together using one mask by the same process as the source electrode 121 and the drain electrode 124, but are not limited thereto.

The first data line DL1 may be disposed on the second insulating layer 104 and covered by the third insulating layer 105. The first data line DL1 may include the same material as the gate electrode 122 and may be formed together using one mask by the same process as the gate electrode 122, but is not limited thereto.

The second data line DL2 may be disposed on the third insulating layer 105 and covered by the fourth insulating layer 106. The second data line DL2 may include the same material as the second storage electrode 142 and may be formed together using one mask by the same process as the second storage electrode 142, but is not limited thereto.

The first data line DL1 may be electrically connected in contact with the first data pad DP1 through the first data contact hole CNT1. The second data line DL2 may be electrically connected in contact with the second data pad DP2 through the second data contact hole CNT2.

The crack prevention pattern CSP may be disposed outside the pad area PA. The crack prevention pattern CSP may be disposed between the ends of the pad area PA and the non-display area NDA2.

However, the plurality of pads VSSP, VDDP, and DP may not be covered by a plurality of inorganic films. The plurality of inorganic films disposed on the fourth insulating layer 106 may expose the plurality of pads VSSP, VDDP, and DP. The plurality of inorganic films disposed on the fourth insulating layer 106 may not be disposed in the pad area PA.

Accordingly, the flexible film COF may be configured so that at least a part thereof is disposed to overlap the pad area PA and attached to the display panel 100, and the flexible film COF may be electrically connected in contact with the plurality of pads VSSP, VDDP, and DP of the pad area PA.

Hereinafter, other embodiments of the present specification will be described. For contents that are substantially the same as those described with reference to FIGS. 1 to 11 among components included in other embodiments, the same reference numerals are given, and overlapping contents may be omitted or briefly described.

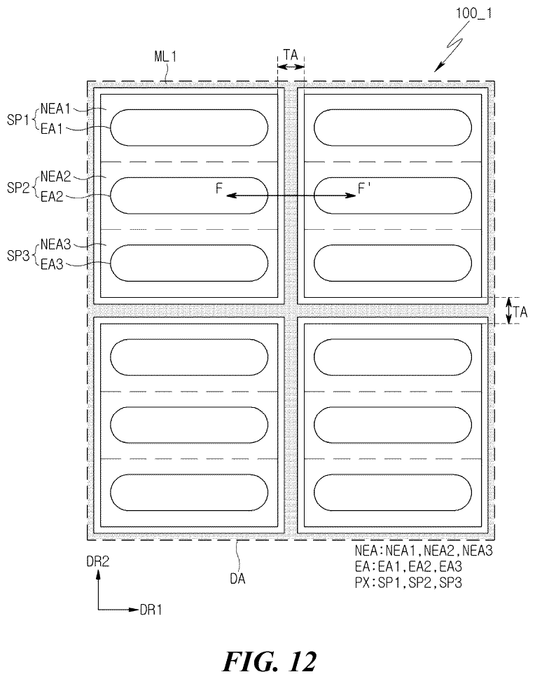

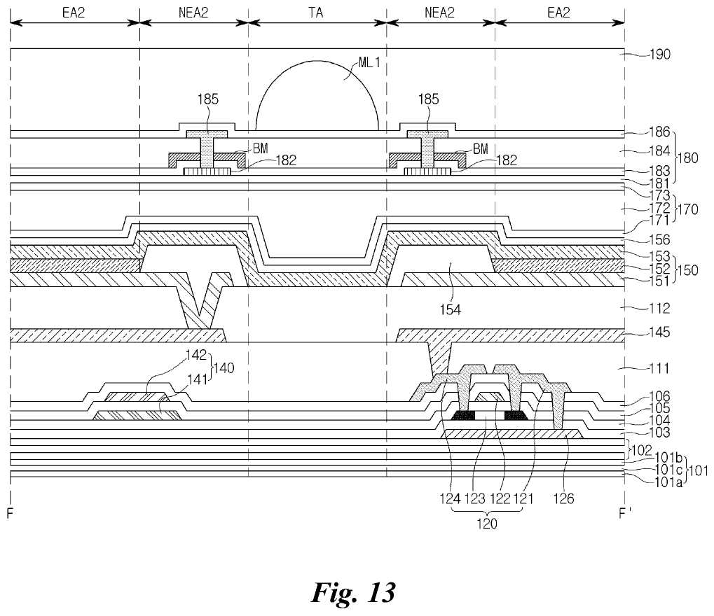

FIG. 12 is an enlarged plan view illustrating a periphery of a pixel of a display apparatus according to another embodiment. FIG. 13 is a cross-sectional view along line F-F′ in FIG. 12.

Referring to FIGS. 12 and 13, a display panel 100_1 of the display apparatus according to the present embodiment may include the first microlens ML1, but may not include the second microlens ML2 (see FIG. 4).

In the display panel 100_1 according to the present embodiment, the first microlens ML1 may be disposed in the transmissive area TA, but the second microlens ML2 (see FIG. 4) may not be disposed in each sub-pixel SP1, SP2, or SP3 of each pixel PX.

The first microlens ML1 of the display panel 100_1 according to the present embodiment may be disposed to extend along the transmissive area TA.

Even in this case, since the first microlens ML1 is disposed in the transmissive area TA, the first microlens ML1 may condense external light incident on the display panel 100_1 into the transmissive area TA, thereby increasing the amount of light passing through the display panel 100_1.

Since the transmissive area TA is disposed in the display area DA, the light transmittance of the transmissive area TA can be increased, and the shape, size, arrangement, etc., of the light-receiving sensor 200 (see FIG. 1) may be designed in various ways. Furthermore, it is possible to secure the path of light incident on the light-receiving sensor 200 and more efficiently perform the process, thereby reducing production energy.

In addition, since the second microlens ML2 is omitted, it is possible to reduce the cost required for the process and further simplify the process, thereby increasing process efficiency.

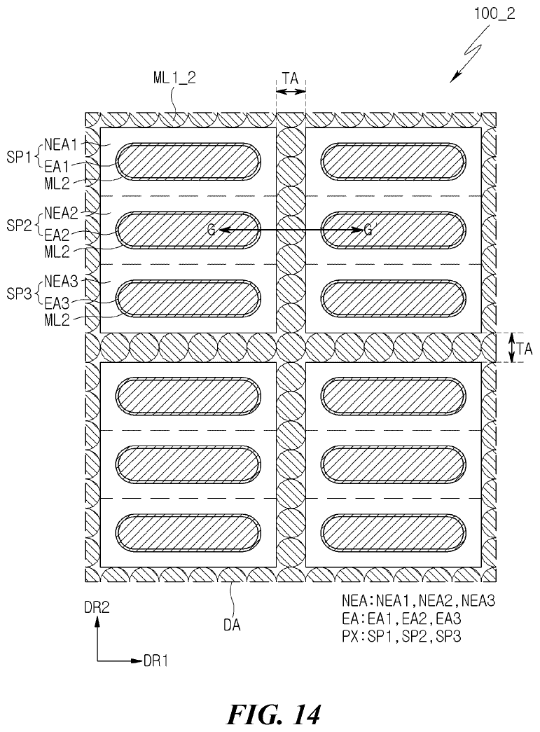

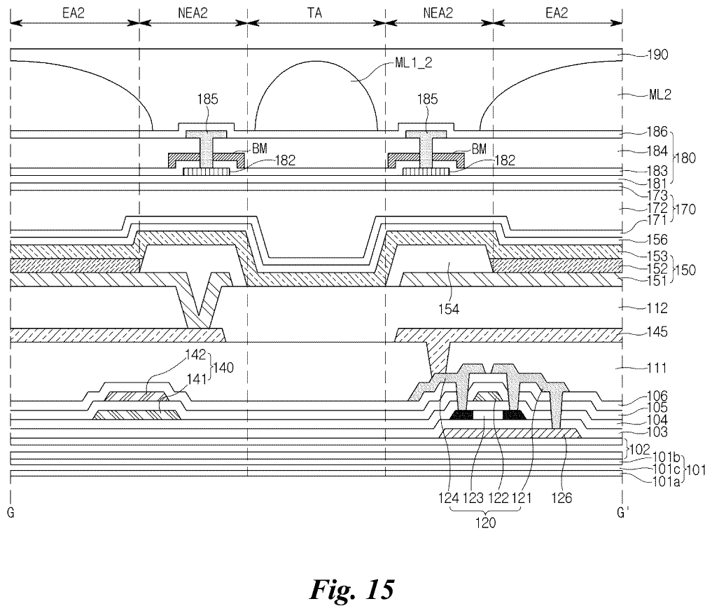

FIG. 14 is an enlarged plan view illustrating a periphery of a pixel of a display apparatus according to another embodiment. FIG. 15 is a cross-sectional view along line F-F′ in FIG. 14.

Referring to FIGS. 14 and 15, a display panel 100_2 of the display apparatus according to the present embodiment includes a first microlens ML1_2 and the second microlens ML2, but the first microlens ML1_2 may be provided as a plurality of first microlenses.

The display panel 100_2 may include the plurality of first microlenses ML1_2. The plurality of first microlenses ML1_2 may be repeatedly disposed in an extension direction of the transmissive area TA. For example, when the transmissive area TA extends in the first direction DR1 and the second direction DR2 between the pixels PX, the plurality of first microlenses ML1_2 may be disposed in the transmissive area TA and repeatedly disposed in the first direction DR1 and the second direction DR2. For example, wherein the plurality of pixels (e.g., SP1, SP2, SP3, . . . ) is arranged in a pixel matrix in a plan view (see FIGS. 4, 12, and 14). Here, the transmissive area TA extends continuously in both a row direction (e.g., pixel row direction) and a column direction (e.g., pixel column direction) of the pixel matrix. As illustrated, the first microlens ML1 extend continuously in a direction parallel to the pixel row or the pixel column of the pixel matrix.

Each of the first microlenses ML1_2 may have a rounded surface. For example, the surface of the first microlens ML1_2 may have a dome shape, but is not limited thereto. The first microlens ML1_2 may have a rounded shape in a cross-sectional view. For example, the first microlens ML1_2 may have a hemispherical or semi-elliptical shape in a cross-sectional view, but is not limited thereto.

Even in this case, since the first microlens ML1_2 is disposed in the transmissive area TA, the first microlens ML1_2 may condense external light incident on the display panel 100_2 into the transmissive area TA, thereby increasing the amount of light passing through the display panel 100_2.

In addition, since each of the plurality of first microlenses ML1_2 is formed in a dome shape, it is possible to more smoothly condense light incident on the display panel 100_2 in various directions, thereby further increasing the amount of light passing through the display panel 100_2.

Since the transmissive area TA is disposed in the display area DA, the light transmittance of the transmissive area TA can be increased, and the shape, size, arrangement, etc., of the light-receiving sensor 200 (see FIG. 1) may be designed in various ways. Furthermore, it is possible to secure the path of light incident on the light-receiving sensor 200 and more efficiently perform the process, thereby reducing production energy.

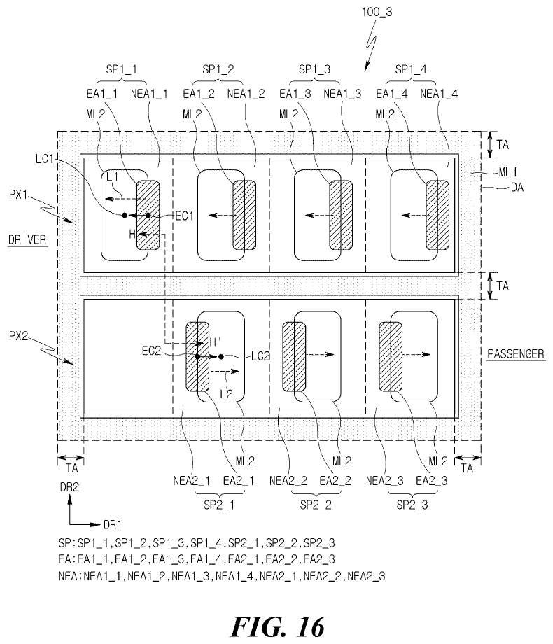

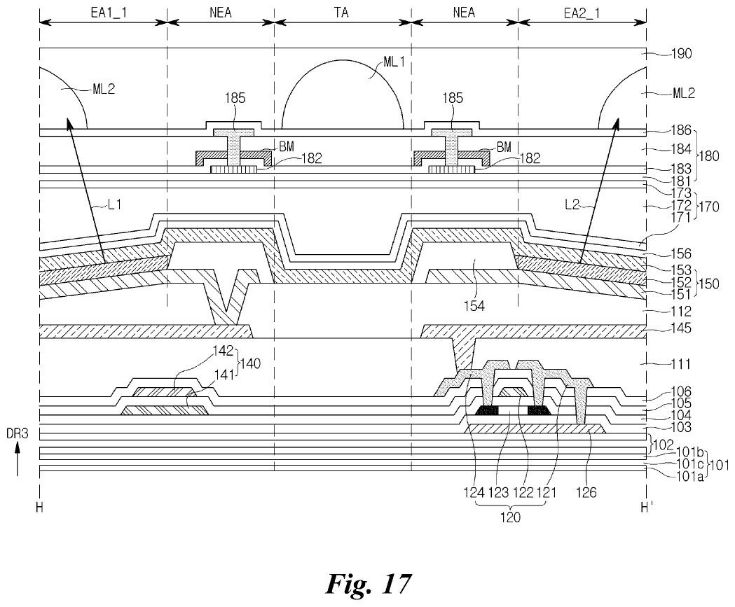

FIG. 16 is an enlarged plan view illustrating a periphery of a pixel of a display apparatus according to yet another embodiment. FIG. 17 is a cross-sectional view along line H-H′ in FIG. 16.

Referring to FIGS. 16 and 17, a display panel 100_3 of the display apparatus according to the present embodiment may include the first microlens ML1 and the second microlens ML2, and centers EC1 and EC2 of the light-emitting area EA and centers LC1 and LC2 of the second microlens ML2 may be misaligned. In addition, each pixel PX1 or PX2 may emit light L1 or L2 in a different direction.

The display panel 100_3 may include a first pixel PX1 and a second pixel PX2 in the display area DA.

Each of the first pixel PX1 and the second pixel PX2 may be disposed repeatedly in the first direction DR1. The first pixel PX1 and the second pixel PX2 may be alternately disposed repeatedly in the second direction DR2.

The sub-pixel SP may include a 1_1 sub-pixel SP1_1, a 1_2 sub-pixel SP1_2, a 1_3 sub-pixel SP1_3, a 1_4 sub-pixel SP1_4, a 2_1 sub-pixel SP2_1, a 2_2 sub-pixel SP2_2, and a 2_3 sub-pixel SP2_3.

The first pixel PX1 may include the 1_1 sub-pixel SP1_1, the 1_2 sub-pixel SP1_2, the 1_3 sub-pixel SP1_3, and the 1_4 sub-pixel SP1_4. The 1_1 sub-pixel SP1_1, the 1_2 sub-pixel SP1_2, the 1_3 sub-pixel SP1_3, and the 1_4 sub-pixel SP1_4 may be disposed in a row in the first direction.

The 1_1 sub-pixel SP1_1 may emit red (R) light, the 1_2 sub-pixel SP1_2 may emit green (G) light, the 1_3 sub-pixel SP1_3 may emit blue (B) light, and the 1_4 sub-pixel SP1_4 may emit red (R) light.

The 1_1 sub-pixel SP1_1, the 1_2 sub-pixel SP1_2, the 1_3 sub-pixel SP1_3, and the 1_4 sub-pixel SP1_4 may include light-emitting areas EA1_1, EA1_2, EA1_3, and EA1_4, and non-light-emitting areas NEA1_1, NEA1_2, NEA1_3, and NEA1_4 disposed around the light-emitting areas EA1_1, EA1_2, EA1_3, and EA1_4, respectively.

The 1_1 sub-pixel SP1_1 may include a 1_1 light-emitting area EA1_1, and a 1_1 non-light-emitting area NEA1_1 disposed around the 1_1 light-emitting area EA1_1.

The 1_2 sub-pixel SP1_2 may include a 1_2 light-emitting area EA1_2, and a 1_2 non-light-emitting area NEA1_2 disposed around the 1_2 light-emitting area EA1_2.

The 1_3 sub-pixel SP1_3 may include a 1_3 light-emitting area EA1_3, and a 1_3 non-light-emitting area NEA1_3 disposed around the 1_3 light-emitting area EA1_3.

The 1_4 sub-pixel SP1_4 may include a 1_4 light-emitting area EA1_4, and a 1_4 non-light-emitting area NEA1_4 disposed around the 1_4 light-emitting area EA1_4.

The second pixel PX2 may include the 2_1 sub-pixel SP2_1, the 2_2 sub-pixel SP2_2, and the 2_3 sub-pixel SP2_3. The 2_1 sub-pixel SP2_1, the 2_2 sub-pixel SP2_2, and the 2_3 sub-pixel SP2_3 may be disposed in a row in the second direction.

The 2_1 sub-pixel SP2_1 may emit blue (B) light, the 2_2 sub-pixel SP2_2 may emit red (R) light, and the 2_3 sub-pixel SP2_3 may emit green (G) light.

The 2-1 sub-pixel SP2_1, the 2_2 sub-pixel SP2_2, and the 2_3 sub-pixel SP2_3 may include light-emitting areas EA2_1, EA2_2, and EA2_3, and non-light-emitting areas NEA2_1, NEA2_2, and NEA2_3 disposed around the light-emitting areas EA2_1, EA2_2, and EA2_3.

The 2_1 sub-pixel SP2_1 may include a 2_1 light-emitting area EA2_1, and a 2_1 non-light-emitting area NEA2_1 disposed around the 2_1 light-emitting area EA2_1.

The 2_2 sub-pixel SP2_2 may include a 2_2 light-emitting area EA2_2, and a 2_2 non-light-emitting area NEA2_2 disposed around the 2_2 light-emitting area EA2_2.

The 2_3 sub-pixel SP2_3 may include a 2_3 light-emitting area EA2_3, and a 2_3 non-light-emitting area NEA2_3 disposed around the 2_3 light-emitting area EA2_3.

In a plan view, no sub-pixel may be disposed below (at the other side in the second direction DR2 of) the 1_1 sub-pixel SP1_1. However, the embodiments of the present specification are not limited thereto, and a sub-pixel may be disposed.

In a plan view, the 2_1 sub-pixel SP2_1 may be disposed below (at the other side in the second direction DR2 of) the 1_2 sub-pixel SP1_2.

In a plan view, the 2_2 sub-pixel SP2_2 may be disposed below (at the other side in the second direction DR2 of) the 1_3 sub-pixel SP1_3.

In a plan view, the 2_3 sub-pixel SP2_3 may be disposed below (at the other side in the second direction DR2) the 1_4 sub-pixel SP1_4.

The transmissive area TA may be disposed between adjacent pixels PX1 and PX2. The transmissive area TA may be disposed between the non-light-emitting areas NEA of adjacent pixels PX1 and PX2.

A light transmittance of the transmissive area TA may be higher than a light transmittance of areas in which the pixels PX1 and PX2 are disposed.

The first microlens ML1 may be disposed in the transmissive area TA. The first microlens ML1 may condense external light entering the display panel 100_3 and increase the amount of light transmitting through the display panel 100_3.

The second microlens ML2 may be disposed on each sub-pixel PX1 or PX2. The second microlens ML2 may be disposed in each sub-pixel SP (SP1_1, SP1_2, SP1_3, SP1_4, SP2_1, SP2_2, or SP2_3).

One second microlens ML2 is illustrated as being disposed in each sub-pixel SP, but the embodiments of the present specification are not limited thereto. For example, according to a design of each sub-pixel SP, the second microlens ML2 disposed in each sub-pixel SP may be provided as two or more microlenses.

The centers LC1 and LC2 of the second microlens ML2 may be misaligned with the centers EC1 and EC2 of the light-emitting area EA corresponding thereto. Nevertheless, some components of the light-emitting part 150 may be tilted, and thus light emitted from the light-emitting area EA of each sub-pixel SP may travel toward the second microlens ML2.

The second protective layer 112 may be formed so that a part of the upper surface thereof has an inclined shape. The inclined surface of the second protective layer 112 may be tilted in a thickness direction (a third direction DR3) of the display panel 100_3.

At least a part of the light-emitting part 150 may be disposed on the inclined surface of the second protective layer 112. Accordingly, at least a part of each of the anode electrode 151 and the organic layer 152 may be inclined (or tilted). At least a part of each of the anode electrode 151 and the organic layer 152 may be tilted toward each of the second microlenses ML2 in each pixel PX1 or PX2.

Specifically, each of the anode electrode 151 and the organic layer 152 may be disposed on the second protective layer 112 of which at least a part is inclined. Each of the anode electrode 151 and the organic layer 152 may be disposed on the second protective layer 112 of which the entire area is inclined, but is not limited thereto.

The anode electrode 151 and the organic layer 152 that are disposed on the inclined second protective layer 112 may be disposed to be inclined (tilted) corresponding to the inclined second protective layer 112. Accordingly, a part of the cathode electrode 153 disposed on the organic layer 152 may be disposed to be inclined.

For example, the anode electrode 151 and the organic layer 152 may be disposed to be inclined in the thickness direction (the third direction DR3) of the display panel 100_3 in the 1_1 light-emitting area EA1_1, the 2_1 light-emitting area EA2_1, and peripheries thereof. That is, a direction in which an upper surface of the anode electrode 151 and an upper surface of the organic layer 152 face may be tilted with respect to the thickness direction (the third direction DR3) of the display panel 100_3.

Accordingly, light emitted from each sub-pixel SP may be tilted with respect to the thickness direction (the third direction DR3) of the display panel 100_3.

In the first light-emitting area EA1_1, the second light-emitting area EA2_1, and peripheries thereof, the directions in which the anode electrode 151 and the organic layer 152 are inclined may be different.

In FIG. 17, the anode electrode 151 and the organic layer 152 around the 1_1 light-emitting area EA1_1 of the 1_1 sub-pixel SP1_1 and the 2_1 light-emitting area EA2_1 of the 2_1 sub-pixel SP2_1 have been described, but the descriptions thereof may be applied to all of the sub-pixels SP.

The center EC1 of the 1_1 light-emitting area EA1_1 of the 1_1 sub-pixel SP1_1 and the center LC1 of the second microlens ML2 disposed on the 1_1 sub-pixel SP1_1 may be misaligned. In a plan view, the center LC1 of the second microlens ML2 may be misaligned from the center EC1 of the 1_1 light-emitting area EA1_1 to the other side (the left side in a plan view) in the first direction DR1.

The description of the misalignment of the 1_1 sub-pixel SP1_1 may also be applied to the remaining sub-pixels SP1_2, SP1_3, and SP1_4 of the first pixel PX1 in substantially the same manner. However, in each of the sub-pixels SP1_1, SP1_2, SP1_3, and SP1_4 of the first pixel PX1, the degree of misalignment between the second microlens ML2 and the light-emitting area EA may be different.

However, the embodiments of the present specification are not limited thereto, and a direction in which the center LC1 of the second microlens ML2 and the center EC1 of the 1_1 light-emitting area EA1_1 are misaligned may vary according to a design.

The center EC2 of the 2_1 light-emitting area EA2_1 of the 2_1 sub-pixel SP2_1 and the center LC2 of the second microlens ML2 disposed on the 2_1 sub-pixel SP2_1 may be misaligned. In a plan view, the center LC2 of the second microlens ML2 may be misaligned from the center EC2 of the 2_1 light-emitting area EA2_1 to one side (the right side in a plan view) in the first direction DR1.

The description of the misalignment of the 2_1 sub-pixel SP2_1 may also be applied to the remaining sub-pixels SP2_2 and SP2_3 of the second pixel PX2 in substantially the same manner. However, in each of the sub-pixels SP2_1, SP2_2, and SP2_3 of the second pixel PX2, the degree of misalignment between the second microlens ML2 and the light-emitting area EA may be different.

However, the embodiments of the present specification are not limited thereto, and a direction in which the center LC2 of the second microlens ML2 and the center EC2 of the 2_1 light-emitting area EA2_1 are misaligned may vary according to a design.

The opening (or the light-emitting area EA) of the sub-pixel SP and the light-emitting part 150 disposed around the opening may be disposed to be tilted with respect to the thickness direction (the third direction DR3), and the light L1 and L2 emitted from the light-emitting part 150 may travel in a direction tilted with respect to the thickness direction (the third direction DR3).

Since the second microlens ML2 and the light-emitting area EA are misaligned, even when light L1 and L2 emitted from the light-emitting part 150 travels while being tilted with respect to the thickness direction (the third direction DR3), each light L1 or L2 may travel toward the second microlens ML2.

The sub-pixels SP1_1, SP1_2, SP1_3, and SP1_4 disposed in the first pixel PX1 may emit the light L1 to the left (the other side in the first direction DR1) in a plan view. The sub-pixels SP2_1, SP2_2, and SP2_3 disposed in the second pixel PX2 may emit the light L2 to the right (one side in the first direction DR1) in a plan view.

That is, the light L1 emitted from the sub-pixels SP1_1, SP1_2, SP1_3, and SP1_4 of the first pixel PX1 may travel while being tilted to the other side in the first direction DR1 with respect to the thickness direction (the third direction DR3). The light L2 emitted from the sub-pixels SP2_1, SP2_2, and SP2_3 of the second pixel PX2 may travel while being tilted to one side in the first direction DR1 with respect to the thickness direction (the third direction DR3).

The direction and degree of misalignment of the second microlens ML2 and the light-emitting area EA may vary according to a traveling direction of the light emitted from each sub-pixel SP of each pixel PX1 or PXG2.

In a plan view, the sub-pixels SP1_1, SP1_2, SP1_3, and SP1_4 disposed in the first pixel PX1 and the sub-pixels SP2_1, SP2_2, and SP2_3 disposed in the second pixel PX2 may emit light in different directions, and thus a screen displayed to a driver DRIVER sitting on the driver's seat and a screen displayed to a passenger PASSENGER sitting on the passenger's seat may be distinctly controlled separately, and different screens may be displayed to the driver DRIVER and the passenger PASSENGER.

Even in this case, since the first microlens ML1 is disposed in the transmissive area TA, the first microlens ML1 may condense external light incident on the display panel 100_3 into the transmissive area TA, thereby increasing the amount of light passing through the display panel 100_3.

Since the transmissive area TA is disposed in the display area DA, the light transmittance of the transmissive area TA can be increased, and the shape, size, arrangement, etc., of the light-receiving sensor 200 (see FIG. 1) may be designed in various ways. Furthermore, it is possible to secure the path of light incident on the light-receiving sensor 200 and more efficiently perform the process, thereby reducing production energy.

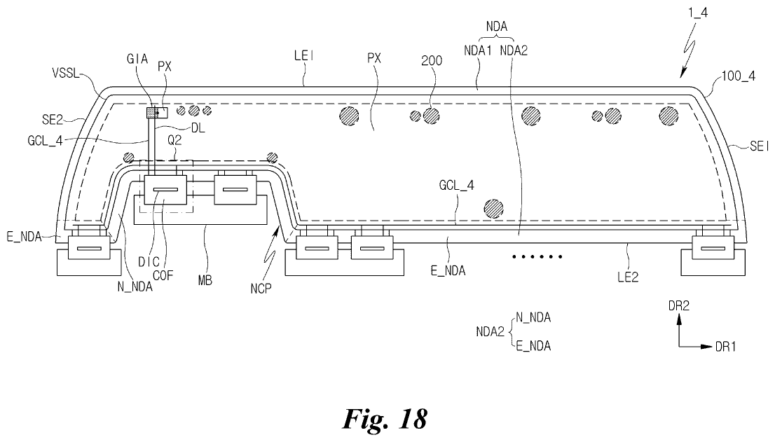

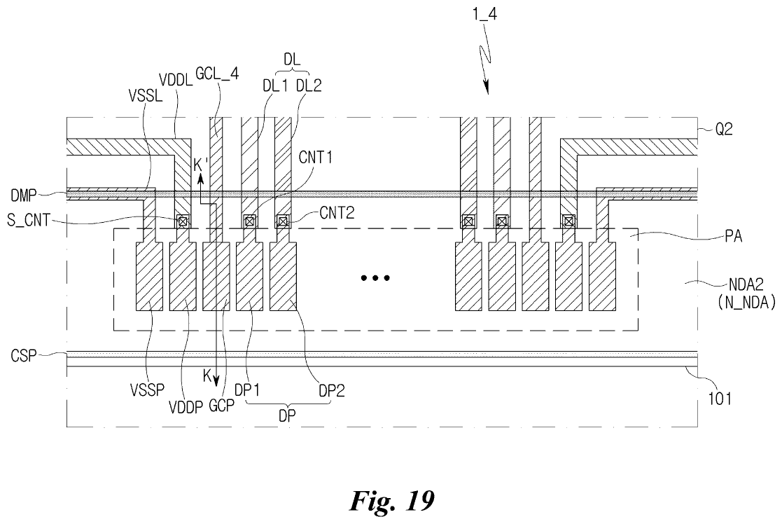

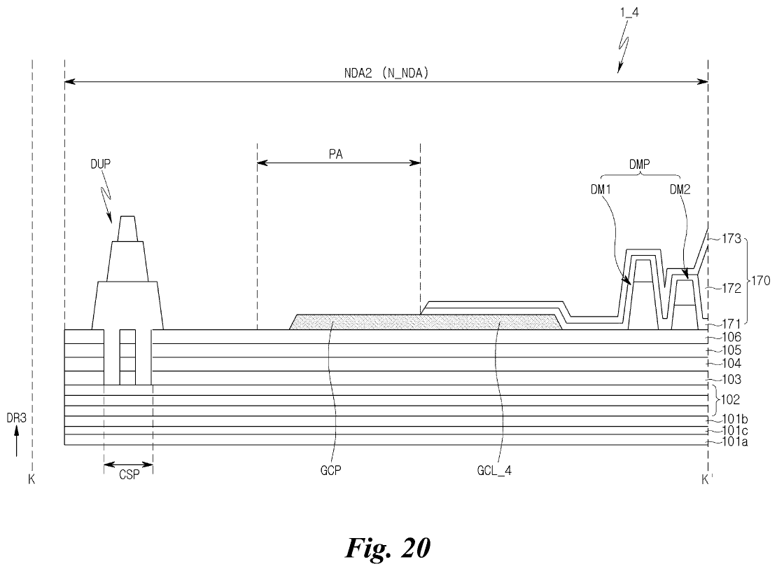

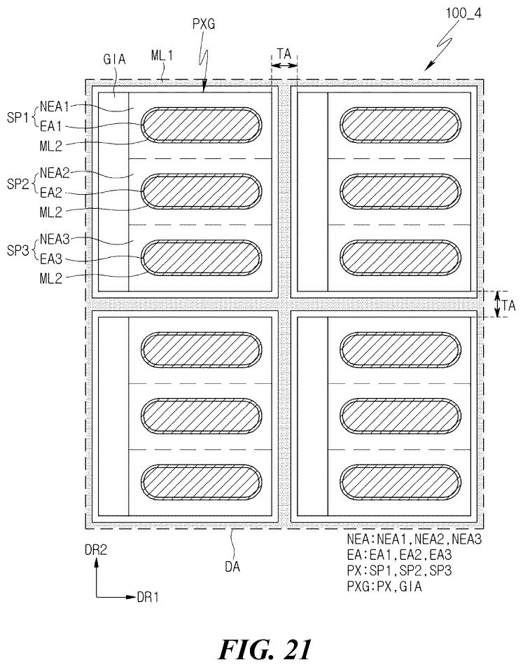

FIG. 18 is a plan view of a display apparatus according to yet another embodiment. FIG. 19 is an enlarged view of area Q2 in FIG. 18. FIG. 20 is a cross-sectional view along line K-K′ in FIG. 19. FIG. 21 is a plan view illustrating arrangement of a pixel of the display apparatus according to yet another embodiment.

FIG. 19 is a view of area Q2 of a display apparatus 1_4 according to yet another embodiment, from which the flexible film COF, the main board MB, and the drive IC DIC are omitted.

Referring to FIGS. 18 to 21, in the display apparatus 1_4 according to the present embodiment, a separate gate driving unit GIP (see FIG. 1) may not be disposed in the non-display area NDA, and a pixel gate driving circuit GIA (also referred to as ‘a pixel gate driving unit GIA’) may be disposed in the display area DA.

The pixel gate driving unit GIA may be provided as a plurality of pixel gate drivers, and each pixel gate driving unit GIA may be connected to each of the plurality of pixels PX. The pixel gate driving unit GIA may be disposed around the pixel PX. The pixel gate driving unit GIA may be disposed between adjacent pixels PX.

For example, the pixel gate driving unit GIA may be disposed between adjacent pixels PX in the first direction DR1. The pixel PX and the pixel gate driving unit GIA may be alternately repeatedly disposed in the first direction DR1. The pixel PX may be continuously repeatedly disposed in the second direction DR2. The pixel gate driving unit GIA may be continuously repeatedly disposed in the second direction DR2.

The pixel gate driving unit GIA may perform substantially the same role as the gate driving unit GIP (see FIG. 1). The pixel gate driving unit GIA may include at least one transistor.

The pixel gate driving unit GIA may be electrically connected to an adjacent pixel PX.

The pixel gate driving unit GIA may receive a gate control signal from the drive IC DIC through a gate control line GCL_4. The pixel gate driving unit GIA may generate a scan signal and a light-emitting signal (or a light-emitting control signal) based on the gate control signal. Accordingly, the driving of the adjacent pixel PX may be controlled.

Since the pixel gate driving unit GIA is disposed in the display area DA, it is possible to minimize the non-display area NDA or the bezel area, thereby providing improved aesthetic feeling to a user.

The display apparatus 1_4 may further include the gate control line GCL_4 and the gate control pad GCP.

The gate control line GCL_4 may be disposed in the non-display area NDA. The gate control line GCL_4 may be disposed in the second non-display area NDA2, but is not limited thereto. The gate control line GCL_4 may be disposed in an extension direction of the second non-display area NDA2.

The gate control line GCL_4 may be electrically connected to the plurality of pixel gate driving units GIAs disposed in the display area DA.

The gate control pad GCP may be disposed in the pad area PA. In the pad area PA, the gate control pad GCP is illustrated as being disposed between the high-potential voltage pad VDDP and the data pad DP, but is not limited thereto, and the arrangement location of the gate control pad GCP may vary according to a design.

The gate control pad GCP may include the same material as the gate control line GCL_4, but is not limited thereto. The gate control pad GCP and the gate control line GCL_4 may be formed integrally, but are not limited thereto.

The gate control pad GCP and the gate control line GCL_4 may be disposed on the fourth insulating layer 106. The gate control pad GCP and the gate control line GCL_4 may be disposed on the same layer as the source electrode 121 (see FIG. 5) and the drain electrode 124 (see FIG. 5) and may include the same material as the source electrode 121 and the drain electrode 124, and the gate control pad GCP, the gate control line GCL, the source electrode 121, and the drain electrode 124 may be formed together using one mask by the same process, but the embodiments of the present specification are not limited thereto.

The plurality of pads VSSP, VDDP, DP, and GCP may not be covered by the plurality of inorganic films. The plurality of inorganic films disposed on the fourth insulating layer 106 may expose the plurality of pads VSSP, VDDP, DP, and GCP. The plurality of inorganic films disposed on the fourth insulating layer 106 may not be disposed in the pad area PA.

Accordingly, the plurality of pads VSSP, VDDP, DP, and GCP disposed on the fourth insulating layer 106 may be exposed, and the display panel 100 may be adhered to the flexible film COF and electrically connected to the flexible film COF.

The display panel 100 of the display apparatus 1_4 according to the present embodiment may also include the transmissive area TA, the first microlens ML1, and the second microlens ML2.

The display panel 100 may further include a pixel group PXG including the pixel PX and a pixel gate driving unit GIA adjacent to the pixel PX.

The transmissive area TA may be disposed between adjacent pixel groups PXG. A light transmittance of the transmissive area TA may be higher than a light transmittance of an area in which the pixel group PXG is disposed.

The arrangement of the transmissive area TA and the first microlens ML1 is not limited thereto. For example, the transmissive area TA and the first microlens ML1 may further be disposed between the pixel PX and the pixel gate driving unit GIA, which constitutes one pixel group PXG.

The first microlens ML1 may be disposed in the transmissive area TA, and the second microlens ML2 may be disposed in each sub-pixel SP1, SP2, or SP3.

Even in this case, since the first microlens ML1 is disposed in the transmissive area TA, the first microlens ML1 may condense external light incident on the display panel 100_4 into the transmissive area TA, thereby increasing the amount of light passing through the display panel 100_4.

Since the transmissive area TA is disposed in the display area DA, the light

transmittance of the transmissive area TA can be increased, and the shape, size, arrangement, etc., of the light-receiving sensor 200 (see FIG. 1) may be designed in various ways. Furthermore, it is possible to secure the path of light incident on the light-receiving sensor 200 and more efficiently perform the process, thereby reducing production energy.

In addition, since the pixel gate driving unit GIA is disposed in the display area DA, thereby reducing the bezel area and increasing the size of the display area DA.

A display apparatus according to various embodiments of the present specification may be described as follows.

According to embodiments of the present specification, there is provided a display apparatus including a substrate including a display area including a plurality of pixels and a transmissive area between adjacent plurality of pixels, and a non-display area around the display area, a thin film transistor disposed on the substrate, a first protective layer on the thin film transistor, a connection electrode electrically connected to the thin film transistor on the first protective layer, a second protective layer on the connection electrode, a light-emitting part on the second protective layer, and a first microlens on the light-emitting part, in which the first microlens is disposed in the transmissive area.

According to various embodiments of the present specification, the display apparatus may further include a light-receiving sensor disposed under the substrate and overlapping the transmissive area.

According to various embodiments of the present specification, a thin film transistor may not be disposed on the transmissive area.

According to various embodiments of the present specification, the light-emitting part may include an anode electrode disposed in each pixel, an organic layer on the anode electrode, and a cathode electrode on the organic layer, and the anode electrode may not be disposed on the transmissive area.

According to various embodiments of the present specification, each pixel may include a plurality of sub-pixels and further include a second microlens disposed in each of the plurality of sub-pixels on the light-emitting part.

According to various embodiments of the present specification, the first microlens and the second microlens may be disposed on the same layer.

According to various embodiments of the present specification, the display apparatus may further a bank between the anode electrode and the organic layer, in which the bank may not be disposed on the transmissive area.

According to various embodiments of the present specification, the bank may be disposed at a boundary between adjacent sub-pixels.

According to various embodiments of the present specification, the sub-pixel may include a light-emitting area and a non-light-emitting area around the light-emitting area, and a center of the light-emitting area and a center of the second microlens may be misaligned.

According to various embodiments of the present specification, the display apparatus may further include a printed circuit film attached to a pad area of the non-display area of the substrate, in which the printed circuit film may be provided as a plurality of printed circuit films.

According to various embodiments of the present specification, the display apparatus may further include a low-potential voltage line, a high-potential voltage line, and a data line that are electrically connected to the pad area, in which, in a non-display area under the display area, the high-potential voltage line may be located between the low-potential voltage line and the display area.

According to various embodiments of the present specification, the low-potential voltage line may surround the display area.

According to various embodiments of the present specification, the non-display area located at a left or right side of the display area may further include a gate driving unit between the low-potential voltage line and the display area.

According to various embodiments of the present specification, the display apparatus may further include a pixel gate driving unit located in the display area and a gate control line electrically connecting the pad area to the pixel gate driving unit.

According to various embodiments of the present specification, the gate control line may be located between the low-potential voltage line and the display area.

According to various embodiments of the present specification, the display apparatus may further include an encapsulation part disposed on the light-emitting part, in which the encapsulation part may include a first inorganic encapsulation layer on the light-emitting part, an organic encapsulation layer on the first inorganic encapsulation layer, and a second inorganic encapsulation layer on the organic encapsulation layer.

According to various embodiments of the present specification, the display apparatus may further include a dam part disposed in the non-display area and overlapping the low-potential voltage line, in which the organic encapsulation layer may be ended inside the dam part.

According to various embodiments of the present specification, the display apparatus may further include a crack prevention pattern disposed between an end portion of the substrate and the dam part.

According to embodiments of the present specification, there is provided a display apparatus including a substrate including a display area including a plurality of pixels and a transmissive area between adjacent plurality of pixels, and a non-display area around the display area, a thin film transistor disposed on the substrate, a first protective layer on the thin film transistor, a light-emitting part on the first protective layer, a first microlens on the light-emitting part, and a light-receiving sensor disposed under the substrate and provided as a plurality of light-receiving sensors, in which the first microlens is disposed in the transmissive area, and at least a part of the light-receiving sensor overlaps the transmissive area.

According to embodiments of the present specification, there is provided a vehicle-integrated display system that incorporates both display and sensing functionalities using specialized optical structures. Specifically, the specification describes a display panel including a substrate having a display area populated by pixels, a non-display area adjacent to the display area, and a transmissive area formed between adjacent pixels, which is intentionally left free of thin film transistors and light-emitting parts to enable light transmission. The light-receiving sensor is disposed below the substrate and is arranged to overlap with the transmissive area in a plan view, allowing external light that has passed through the transmissive area to be captured by the sensor. This structural configuration is described in the context of enhancing sensing functions such as gesture recognition, ambient light detection, or camera input, while maintaining pixel density and display integrity. Furthermore, the specification discloses that microlenses may be formed in the transmissive area to condense or direct light. The first microlens is positioned to focus external light toward the underlying sensor, increasing sensing efficiency. Additionally, the specification describes arrangements wherein a second microlens is provided on the same layer as the first microlens and is configured to control the emission direction of display light—e.g., redirecting light toward a vehicle driver or passenger—thereby improving visibility or privacy based on seating position.

According to embodiments of the present specification, there is provided a method. In one embodiment, a method of sensing light through a display panel is provided. The method includes condensing external light through a microlens disposed in a transmissive area of a display panel. The transmissive area may be formed between adjacent pixels in a display area of the display panel, and may be configured without a thin film transistor or light-emitting component. The microlens may be disposed in a layer above or within the display stack and configured to converge incident external light toward a focal region.

The method further includes transmitting the condensed external light through the display panel to a light-receiving sensor. The condensed external light passes through the transmissive area of the display panel without obstruction by circuit elements. In some embodiments, the transmissive area comprises one or more transparent planarization layers, an upper substrate, and other transparent or semi-transparent elements of the display stack. In some embodiments, the transmissive area is formed without a through-hole penetrating the display panel.

The method further includes detecting the condensed external light at the light-receiving sensor. The light-receiving sensor may be disposed below the substrate of the display panel and at least partially overlapping the transmissive area in a plan view. The sensor may be configured to detect light for purposes including, but not limited to, user detection, ambient light sensing, or biometric sensing. By condensing, transmitting, and detecting external light in this manner, the display panel can support sensing functionality while maintaining mechanical integrity and avoiding structural discontinuities associated with through-holes.

According to various embodiments of the present specification, the light-emitting part may include an anode electrode disposed in each pixel, an organic layer on the anode electrode, and a cathode electrode on the organic layer, in which the anode electrode may not be disposed on the transmissive area, and each pixel may include a plurality of sub-pixels and further include a second microlens disposed in each of the plurality of sub-pixels on the light emitting part.

Although the embodiments have been described above with reference to the accompanying drawings, those skilled in the art to which the present specification pertains will be able to understand that the above-described technical configuration can be carried out in other specific forms without changing the technical spirit or essential features thereof. Accordingly, it should be understood that the above-described embodiments are illustrative and not restrictive in all respects. In addition, the scope of the embodiments is determined by the appended claims rather than detailed description. In addition, the meaning and scope of the claims and all changed or modified forms derived from the equivalent concept thereof should be construed as being included in the scope of the embodiments.

### DESCRIPTION OF REFERENCE NUMERALS

1: display apparatus
100: display apparatus
200: light-receiving sensor
101: substrate
120: thin film transistor
150: light emitting part
180: encapsulation part
ML1: first microlens
ML2: second microlens
NCP: notch
DA: display area
NDA: non-display area
NDA1: first non-display area
NDA2: second non-display area
N_NDA: notch non-display area
E_NDA: extension non-display area
PA: pad area
PX: pixel
SP: sub-pixel
EA: light-emitting area
NEA: non-light-emitting area
TA: transmissive area

The various embodiments described above can be combined to provide further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A display apparatus comprising:
a substrate including:
a display area including a plurality of pixels;
a transmissive area between adjacent pixels of the plurality of pixels; and
a non-display area adjacent to the display area;
a thin film transistor on the substrate;
a first protective layer on the thin film transistor;
a connection electrode electrically connected to the thin film transistor on the first protective layer;
a second protective layer on the connection electrode;
a light-emitting part on the second protective layer; and
a first microlens on the light-emitting part,
wherein the first microlens is disposed in the transmissive area.

2. The display apparatus of claim 1, further comprising a light-receiving sensor disposed under the substrate and overlapping the transmissive area.

3. The display apparatus of claim 1, wherein the thin film transistor is not disposed on the transmissive area.

4. The display apparatus of claim 1, wherein the light-emitting part includes an anode electrode disposed in each pixel, an organic layer on the anode electrode, and a cathode electrode on the organic layer, and
wherein the anode electrode is not disposed on the transmissive area.

5. The display apparatus of claim 4, wherein each of the pixels includes a plurality of sub-pixels, and
further includes a second microlens disposed in each of the plurality of sub-pixels on the light-emitting part.

6. The display apparatus of claim 5, wherein the first microlens and the second microlens are on a same layer.

7. The display apparatus of claim 5, further comprising a bank between the anode electrode and the organic layer,
wherein the bank is not disposed on the transmissive area.

8. The display apparatus of claim 7, wherein the bank is located at a boundary between adjacent sub-pixels of the plurality of sub-pixels.

9. The display apparatus of claim 5, wherein the sub-pixel includes a light-emitting area, and a non-light-emitting area adjacent to the light-emitting area, and
wherein a center of the light-emitting area and a center of the second microlens are misaligned.

10. The display apparatus of claim 1, further comprising a printed circuit film attached to a pad area of the non-display area of the substrate, in which the printed circuit film is provided as a plurality of printed circuit films.

11. The display apparatus of claim 10, further comprising a low-potential voltage line, a high-potential voltage line, and a data line that are electrically connected to the pad area,
wherein, in the non-display area under the display area, the high-potential voltage line is located between the low-potential voltage line and the display area.

12. The display apparatus of claim 11, wherein the low-potential voltage line surrounds the display area.

13. The display apparatus of claim 12, wherein the non-display area located at either a left side or a right side of the display area further includes a gate driving unit between the low-potential voltage line and the display area.

14. The display apparatus of claim 12, further comprising: a pixel gate driving circuit located in the display area; and
a gate control line electrically connecting the pad area to the pixel gate driving circuit.

15. The display apparatus of claim 14, wherein the gate control line is located between the low-potential voltage line and the display area.

16. The display apparatus of claim 11, further comprising an encapsulation part on the light-emitting part,
wherein the encapsulation part includes a first inorganic encapsulation layer on the light-emitting part, an organic encapsulation layer on the first inorganic encapsulation layer, and a second inorganic encapsulation layer on the organic encapsulation layer.

17. The display apparatus of claim 16, further comprising a dam part disposed in the non-display area and overlapping the low-potential voltage line, wherein the organic encapsulation layer is ended inside the dam part.

18. The display apparatus of claim 17, further comprising a crack prevention pattern disposed between an end portion of the substrate and the dam part.

19. A display apparatus comprising:
a substrate including:
a display area including a plurality of pixels;
a transmissive area between adjacent pixels of the plurality of pixels; and
a non-display area adjacent to the display area;
a thin film transistor on the substrate;
a first protective layer on the thin film transistor;
a light-emitting part on the first protective layer;
a first microlens on the light-emitting part; and
a light-receiving sensor disposed under the substrate and provided as a plurality of light-receiving sensors,
wherein the first microlens is disposed in the transmissive area, and
wherein at least a part of the light-receiving sensor overlaps the transmissive area.

20. The display apparatus of claim 19, wherein the light-emitting part includes an anode electrode disposed in each pixel, an organic layer on the anode electrode, and a cathode electrode on the organic layer,
wherein the anode electrode is not disposed on the transmissive area, and
wherein each of the pixels includes a plurality of sub-pixels, and
further includes a second microlens disposed in each of the plurality of sub-pixels on the light-emitting part.

21. A display apparatus comprising:
a substrate including:
a display area including a plurality of pixels; and
a transmissive area between adjacent pixels of the plurality of pixels;
a thin film transistor on the substrate;
a light-emitting part on the thin film transistor in the display area
a microlens in the transmissive area; and
a light-receiving sensor disposed adjacent to the substrate and overlapping the transmissive area in a plan view,
wherein the transmissive area excludes the thin film transistor and the light-emitting part.

22. The display apparatus of claim 21, further comprising: a lens protective film on the microlens, the lens protective film having a lower refractive index than the microlens.

23. The display apparatus of claim 21, further comprising a bank layer adjacent to the light-emitting part, and
wherein the transmissive area does not overlap with the bank layer in a plan view.

24. The display apparatus of claim 21, wherein the microlens extends in a direction of the transmissive area.

25. The display apparatus of claim 21, wherein the plurality of pixels is arranged in a pixel matrix in a plan view, and
wherein the transmissive area extends continuously in both a row direction and a column direction of the pixel matrix.

26. A display apparatus comprising:
a substrate including:
a display area including a plurality of pixels; and
a transmissive area between adjacent pixels of the plurality of pixels;
a first microlens in the transmissive area, the first microlens, in operation, configured to condense light incident from an external source;
a second microlens on a pixel of the plurality of pixels, the second microlens, in operation, configured to control an emission direction of light from the pixel; and
a light-receiving sensor disposed beneath the transmissive area.

27. The display apparatus of claim 26, wherein the first microlens and second microlens are formed in a same layer.

28. The display apparatus of claim 26, wherein the display area further includes a light-emitting area and a non-light-emitting area, and
wherein the second microlens covers at least a portion of both the light-emitting area and the non-light-emitting area in a plan view.

29. The display apparatus of claim 26, wherein the plurality of pixels is arranged in a pixel matrix in a plan view, and
wherein the first microlens extend continuously in a direction parallel to a pixel row or a pixel column of the pixel matrix.

30. The display apparatus of claim 26, wherein the light-receiving sensor is configured to detect at least one of ambient light, infrared light, or biometric characteristics.

31. A vehicle comprising:
a body having a motor mounted therein;
at least one display panel mounted to the body of the vehicle, the display panel including:
a substrate including a display area having a plurality of pixels, a transmissive area between adjacent pixels of the plurality of pixels, and a non-display area adjacent to the display area;
a thin film transistor on the substrate;
a light-emitting part on the thin film transistor in the display area;
a first microlens disposed in the transmissive area and configured to condense external light; and
a light-receiving sensor disposed below the substrate and at least partially overlapping the transmissive area in a plan view,
wherein the transmissive area excludes the thin film transistor and the light-emitting part.

32. The vehicle of claim 31, further comprising a second microlens adjacent to the first microlens and disposed on a same layer as the first microlens,
wherein the second microlens is configured to direct light emitted from the plurality of pixels toward either a driver seat or a passenger seat.

33. A method comprising:
condensing external light through a microlens disposed in a transmissive area of a display panel;
transmitting the condensed external light through the display panel to a light-receiving sensor disposed below the transmissive area; and
detecting the condensed external light at the light-receiving sensor,
wherein the transmissive area is formed without a through-hole in the display panel.
